(19) United States
(12) Patent Application Publication
(10) Pub. No.: US 2004/0072249 A1
(43) Pub. Date: Apr. 15, 2004

(54) Methods for peptide-protein binding prediction

(76) Inventors: Stephen L. Hoffman, Gaithersburg, MD (US); Sorin Istrail, Rockville, MD (US); Liliana Florea, Washington, DC (US); Bjarni V. Halldorsson, Gaithersburg, MD (US); Oliver Kohlbacher, Tubingen (DE); Russell S. Schwartz, Pittsburgh, PA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/608,915

(22) Filed: Jun. 27, 2003

Related U.S. Application Data
(60) Provisional application No. 60/392,843, filed on Jun. 28, 2002.

(51) Int. Cl. G01N 33/53; G06F 19/00; G01N 33/48; G01N 33/50

(52) U.S. Cl. 435/7.1; 702/19

(57) ABSTRACT

The present teachings are related to methods for predicting the binding affinity of peptides for proteins. Individual methods for creating predictions and methods for combining predictions are also related. Some embodiments of the present teachings include methods for manufacturing a vaccine and a computer system for implementing the methods of the present teachings.

### RELATED APPLICATIONS

This application claims priority to Provisional Application No. 60/392,843, filed on Jun. 28, 2002. The subject matter of the aforementioned application is hereby incorporated by reference in its entirety.

### BACKGROUND OF THE INVENTION

The evolution of the immune systems of vertebrate animals has produced a variety of responses for providing protection against infection and disease. Cytotoxic immune system cells of vertebrates are able to survey the cells of the body and detect those that may be infected or diseased. An important step in this surveillance is the interaction between the Major Histocompatibility Complex (MHC) present on the surface of all cells and the receptors on the surfaces of T cells (T cell receptors, or TCRs). The MHC class I immune response provides a means of monitoring protein expression within cells and inducing lysis in those cells exhibiting aberrant expression. This immune response is crucial to targeting virus-infected cells, which must express non-self peptides during the course of reproducing an infecting virus. The MHC class I immune response also provides one line of defense against cancer, as proteins that are mis-spliced or over-expressed due to genetic damage can trigger an immune response, leading to death of the malignant cells.

Recently it has been proposed to use the MHC class I immune response to fight disease. For example, cancer immunotherapeutics is a recent therapeutic approach aimed at priming the immune system to respond to malignant cells in order to trigger an immune response in patients whose immune systems are not responding on their own (Rosenberg, 1999). Whether the goal is therapy for cancer or viral disease, these approaches require the selection of those peptides that can stimulate the immune system to seek out and destroy infected or diseased cells.

The pathway by which a viral protein or an aberrant self-protein can induce an immune response starts with the protein being cleaved by the proteasome in the cytoplasm to produce individual peptides, which are then transported into the endoplasmic reticulum (ER) by the TAP transporter, and some of them incorporated into the major histocompatibility complex (MHC), presented at the cell surface. At the end, recognition of the MHC by the T cell receptor (TCR) found on the surface of CD8+T cells triggers apoptosis of the presenting cell. The cleavage of proteins into peptide antigens presented on the MHC is not a random process. Each of the steps in the antigen-processing pathway adds some specificity to antigen selection. However, the primary and most discriminating point at which sequence specificity constrains antigen recognition appears to be incorporation of peptides in the MHC complex (Lauemøller et al., 2001).

The geometry of the MHC-peptide binding is relatively well known for class I MHCs, and has been explored by epitope prediction methods. The binding of peptides to the MHC binding groove is specific (Madden et al., 1992), generally at the N-and C-termini of the peptide (Bouvier and Wiley, 1994), although it can extend beyond the groove (Collins et al., 1994). The main source of specificity is the “anchor sites,” which are pockets in the MHC complex that accommodate certain peptide side chains. On the class I MHC, the anchors are near the ends of the peptides, typically on the second and last positions, allowing peptides to bulge out in the intervening region (Guo et al., 1992). The MHC genes are highly variable among human populations, with different HLA alleles conferring different peptide specificities, particularly at the anchor positions. For example, the anchor position specificities for nine-mer peptide antigens binding to the five major MHC class I allotypes: A1, A2, A3, A24, and B7, are given below in Table 1.

TABLE 1
Preferred anchor residues of the HLA supertypes A1, A2, A3, A24,
and B7, as described in Marsh et al. (2000).

| | Preferred Anchor Residues by Position | | | | | | | | |
|---|---|---|---|---|---|---|---|---|---|
| Allotype | 1 | 2 | 3 | 4 | 5 | 6 | 7 | 8 | 9 |

| | | | |
|---|---|---|---|
| A1 | | D, E | Y |
| A2 | L, V, Q | | L, V |
| A3 | L, V, M | | K, Y |
| A24 | Y, F | | F, W, I, L |
| B7 | P | | L |

The pathway from protein sequence to vaccine development is lengthy and cost-intensive, requiring the development of binding assays for testing the affinity of the selected peptide(s) to the MHC molecules, in vitro assays for measuring the T-cell response, and ultimately in vivo testing of immunogenicity. Therefore, careful selection of candidates from the thousands of peptides in the proteins of interest is imperative from the start. Computational immunovaccinology, or computer aided vaccine design, is gaining increased attention from both theoretical bioinformaticists and vaccine experimentalists as a rapid and inexpensive way to screen peptides to identify hits for therapeutic area. With the completion of the human genome sequence and initial characterization of the human protein repertoire (Venter et al., 2001; Lander et al., 2001), the sheer amount of data to be analyzed adds the challenge of scale to the quest for fast and effective screening methods. There exists a need for methods that can quickly select candidate epitope peptides with a high degree of accuracy.

Description of the Related Art

The earliest work attempting to characterize and classify the epitopes of particular MHC proteins focused on identifying and screening for anchor residues in epitope peptides and potential epitopes. For example, early methods for prediction focused on characterizing likely epitopes by testing for the presence of the appropriate primary anchors (Falk et al., 1991; Hunt et al., 1992; DiBrino et al., 1993), and secondary anchor residues (Ruppert et al., 1993). An in silico epitope prediction method based on anchor identification was developed by Rammensee et al. (Rammensee et al., 1999). It produced an algorithm for predicting epitopes from protein sequences, and a database (SYFPEITHI; http://syfpeithi.bmi-heidelberg.com/) of experimentally identified and published motifs, both publicly accessible through a web interface. Elaboration of these techniques lead to the development of EpiMer (De Groot et al., 2001a, which uses a pattern-matching prediction algorithm based on the same principles for identifying peptides that may potentially bind to one or more MHC proteins. Alternatives to these pattern-based methods include neural networks (Gulukota et al., 1997; Milik et al., 1998; Buus, unpublished), statistical methods for parameter estimation (Gulukota et al., 1997), and structure-based methods (Rognan et al., 1994; Altuvia et al., 1997; Rognan et al., 1999; Logean et al., 2001; Schueler-Furman et al., 2001).

### SUMMARY OF THE INVENTION

One aspect of the present teachings relates to a method for predicting the binding affinity of a peptide for a MHC protein. The method comprises providing a prediction of binding affinity by at least three different methods. The predictions of binding affinity from these methods are combined into a single prediction of binding affinity. The method can be used with MHC class I proteins in general as well as with specific MHC class I protein allotypes, such as A1, A2, A3, A24 and B7. Peptides for use with the method include peptides derived from viral or human proteins and peptides whose sequences are derived from genomic sequences. Other embodiments of the invention envisions other sources for the candidate peptides. For example, randomly generated sequences or sequences isolated from the peptidome of an organism could be used as a source for candidate peptide sequences. The combination of the predictions of binding affinity can be produced through a voting method. The predictions of affinity that are produced by the methods can be presented in terms of the relative binding efficiences of peptides, IC-50 values, and categorical binding affinities. Some ways of predicting the affinity of the peptide for the MHC protein include algorithms such as quadratic programs, linear programs, and profile-based methods. Clustering heuristics such as iterative multiple alignment, letter frequencies and position dependencies can be used with profile-based methods of predicting affinity. Principles such as dimensionality reduction, multiple intra-allelic motifs, and anchor selection can also be used with the profile-based methods.

In an additional aspect of the present teachings, methods can assign a categorical binding affinity to a candidate peptide based on an evaluation of the peptide's affinity for a protein. Information about a set of known peptides, including sequence and binding affinity information, is used, along with sequence information about the candidate peptide, to generate an evaluation of the affinity of the candidate peptide for the protein. The candidate peptide can be ranked among the known peptides on the basis of the peptides' binding affinities relative to one another. Peptides can be assigned to categories of binding affinity based on rank.

In some embodiments of this method, the ranking created is a list of known peptides organized by the levels of affinity they have for the protein. In some embodiments, the candidate peptide is a part of this list.

In certain embodiments, the categorical binding affinity that is assigned to a peptide consists of one of two or more classes. This is based on the ranking of the peptides in the list. These classes can be labeled high, medium, low, or non-binder.

Another aspect of the present teachings is a method for evaluating the affinity of a candidate peptide for a protein. This method comprises obtaining information for a set of known peptides, including sequence and binding affinity information, as well as sequence information for the candidate peptide. Data developed using the information for a set of known peptides and a first prediction method is used with the sequence information for the candidate peptide to generate a first evaluation of the binding affinity of the candidate peptide for the protein. This process is repeated one or more times, with different prediction methods, to generate additional evaluations. The evaluations are combined to create a single evaluation of the candidate peptide's binding affinity for the protein.

In some embodiments of this method, the binding affinity information for the set of known peptides is given in quantified affinity data for individual peptides in the set. In other embodiments, the binding affinity information is data regarding whether individual peptides bind to the protein or not. Some embodiments use binding affinity data that comprises the assignments of known peptides in three or more catagories of binding. In some of these embodiments, the catagories are labeled high, medium, low and non-binders. In other embodiments, a combination of the types of binding affinity information disclosed above is used. Some embodiments use prediction methods such as quadratic programming, linear programming, and profile-based methods. Some embodiments that employ profile-based methods can also incorporate methods such as iterative multiple alignment, letter frequencies, positional dependencies based on (2 statistical significance tests, and can implement priniciples such as dimensionality reduction, multiple intra-allelic motifs, and anchor selection. In embodiments that employ quadratic programming as a prediction method, data reduction methods are sometimes used, such as a BIMAS-like method and the AA-properties method.

In some embodiments of the method, sequence data for a candidate peptide is from viral protein sequence data or human protein sequence data. Some embodiments used a centralized database tostore candidate peptide sequence data, sequence and binding affinity information for a set of known peptides, evaluations of candidate peptide binding affinity and/or a combined evaluation of the binding affinity of a candidate peptide for a protein.

In some embodiments, the protein is a MHC protein. In some of such embodiments, the MHC protein is a class I MHC protein. In some of those embodiments, the class I MHC protein is an A1, A2, A3, A24, orB7 allotype.

In particular embodiments of the method, the combined evaluation of the affinity of a candidate peptide of a protein consists of the assignment of a rank to the candidate peptide. In some of these embodiments, the rank is based on the affinity of the candidate peptide for the protein relative to other peptides.

Certain embodiments of the method envision the evaluation of multiple candidate peptides. In some of these embodiments, a subset of candidate peptides is selected based on the combined evaluations of those peptides.

An additional embodiment includes a method of making a vaccine by manufacturing a peptide that was selected using the method and preparing a medicament using the peptide.

Another aspect of the present teachings include an algorithmic quadratic programing method that comprises methods for predicting the relative affinities of potential peptide epitopes for binding to MHC proteins using position-weight matrices and quadratic equations. Some embodiments feature the equation below with the constraints that follow:

$${min}_{w , c} {\sum }_{i}^{} {( {x}_{i}^{T} w - c - {b}_{i} )}^{2}$$

$$s . t . {x}_{{H}_{i}}^{T} w \geq {x}_{{M}_{j}}^{T} w ∀ i , j , \\ {x}_{{H}_{i}}^{T} w \geq {x}_{{L}_{j}}^{T} w ∀ i , j , \\ {x}_{{M}_{i}}^{T} w \geq {x}_{{L}_{j}}^{T} w ∀ i , j , \\ {x}_{{e}_{i}}^{T} w \geq {IC}_{min}^{50} , {x}_{{ne}_{j}}^{T} , w \leq {IC}_{min}^{50}$$

Additional embodiments feature the following equation with constraints:

$${min}_{w , c} {\sum }_{i}^{} {( {x}_{i}^{T} w - c - {b}_{i} )}^{2} + {\sum }_{i , j}^{} {e}_{{H}_{i} {M}_{j}}^{2} + {\sum }_{i , j}^{} {e}_{{H}_{i} {L}_{j}}^{2} + {\sum }_{i , j}^{} {e}_{{M}_{i} {L}_{j}}^{2} + {\sum }_{i , j}^{} {e}_{{e}_{i}}^{2} {\sum }_{i , j}^{} {e}_{{ne}_{i}}^{2}$$

$$s . t . ( {x}_{{H}_{i}}^{T} - {x}_{{M}_{j}}^{T} ) w + {e}_{{H}_{i} {M}_{j}} \geq 0 ∀ i , j \\ ( {x}_{{H}_{i}}^{T} - {x}_{{L}_{j}}^{T} ) w + {e}_{{H}_{i} {L}_{j}} \geq 0 ∀ i , j \\ ( {x}_{{M}_{i}}^{T} - {x}_{{L}_{j}}^{T} ) w + {e}_{{M}_{i} {L}_{j}} \geq 0 ∀ i , j$$

$${x}_{{e}_{i}}^{T} w - {IC}_{min}^{50} + {e}_{{e}_{i}} \geq 0 ∀ i$$

$${IC}_{min}^{50} - {x}_{{ne}_{i}}^{T} w + {e}_{{ne}_{i}} \geq 0 ∀ i$$

In another aspect of the present teachings, linear programming methods can be used to predict the relative binding affinities for a set of peptides. In other aspects, linear programming methods can be used to predict which peptides from a candidate set of peptides are likely to be epitopes for a particular protein.

Some aspects of the present teachings feature a linear programming method featuring the following equation:

$$min {\sum }_{{aa}^{′} sa}^{} {\sum }_{pos . p}^{} {b}_{ap}$$

$$s . t . {\sum }_{pos . p}^{} {b}_{{e}_{i}^{p} p} \geq 1 ∀ i$$

$${b}_{ap} \geq 0 ∀ a , p$$

Additional aspects of the present teachings are profile-based methods for predicting peptide affinity, which comprise obtaining information for a set of known peptides, creating one or more motifs for peptides with affinity to the protein by analyzing the information for the set of known peptides, and evaluating the affinity of peptides for the protein based on the one or more motifs. In some embodiments, the information for a set of known peptides comprises sequence information. In some embodiments, clustering heuristics are employed, such as iterative multiple alignment, letter frequencies, and position dependencies reflected by χ² tests.

In another aspect of the present teachings, an iterative multiple alignment method of predicting the relative binding affinity of a peptide for a MHC protein is used. This method comprises obtaining sequence and affinity information for a set of known epitopes for the MHC protein, deriving one or more motifs from the information with an iterative multiple alignment heuristic, generating a score for the peptide based on its similarity to the derived motif, and predicting the peptide's relative binding affinity for the MHC protein based on its score. In some embodiments, this method is extended to derive multiple motifs from the information using an iterative multiple alignment heuristic and generating a score for the peptide from each of the motifs. In these embodiments, the relative binding affinity of the peptide for the MHC protein is based on the score showing the highest binding affinity.

An additional aspect of the present teachings is a letter frequency method of predicting the relative binding affinity of a peptide for a MHC protein. This method comprises obtaining sequence and affinity information for a set of known epitopes for a MHC protein, deriving one or more motifs from the information with a letter frequency heuristic, generating a score for the peptide based on its similarity to the derived motif, and predicting the relative binding affinity of the peptide for the MHC protein based on its score. An additional embodiment comprises deriving multiple motifs from the information using a letter frequency heuristic, and generating a score for the peptide from each of the motifs. In these embodiments, the relative binding affinity of the peptide for the MHC protein is based on the score showing the highest binding affinity.

Yet another aspect of the present teachings is a χ² statistical significance (Ki2) test method of predicting the relative binding affinity of a peptide for a MHC protein. This method comprises obtaining sequence and affinity information for a set of known epitopes for a MHC protein, deriving one or more motifs from the information according to positional dependencies revealed by a χ² statistical test heuristic, generating a score for the peptide based on its similarity to the derived motif, and predicting the relative binding affinity of the peptide for the MHC protein based on its score. Some embodiments comprise deriving multiple motifs from the information using a χ² statistical test heuristic, and generating a score for the peptide from each of the motifs. In these embodiments, the relative binding affinity of the peptide for the MHC protein is based on the score showing the highest binding affinity.

A method of selecting peptides with desired level of affinity for a protein is another aspect of the present teachings. This method comprises obtaining sequence data for candidate peptides and for peptides with known affinity for a protein and evaluating the affinity of candidate peptides for the protein by two or more affinity evaluation methods. At least one of the evaluation methods uses the data for the peptides with known affinity. The method comprises selecting peptides from the set of candidate peptides that are predicted by the affinity evaluation methods to have the desired level of affinity. In some embodiments, the data for peptides of known affinity includes sequence data and quantitative binding affinity data or qualitative binding data for the binding of the peptides to the protein. In some embodiments, the selected peptides with a desired affinity for the protein are used for the treatment or prevention of disease.

The aspects of the present teachings include a method for providing treatment for a patient with, or at risk for, cancer or a viral infection, comprising obtaining sequence data for candidate peptides and data for a set of known peptides, evaluating the affinity of the candidate peptides for a protein with two or more prediction methods, selecting candidate peptides with a desired level of affinity for the protein for inclusion in a treatment and treating the patient by stimulating an immune response in the patient with the treatment. In some embodiments, the sequence data for the candidate peptides comes from tumor cell proteins or genes or viral proteins or genes. Data for the set of known peptides comprises sequence data and data regarding the affinity of the peptides for the protein in particular embodiments. In some embodiments, an additional step to the method is added, which comprises determining the allotype of the protein by obtaining genetic information about the patient. In certain embodiments, the protein is a MHC protein.

Another aspect of the present teachings is a method for screening a set of candidate peptides, comprising obtaining sequence data for candidate peptides, obtaining data for a set of known peptides, evaluating the affinity of the candidate peptides for a protein with two or more prediction methods, assigning the candidate peptides to two or more groups based on the evaluations of affinity, and screening the set of candidate peptides based on the group assignment. In certain embodiments, the sequence data for the candidate peptides is selected from sequence data of a known protein, sequence data from a set of known proteins or genomic sequences. In some embodiments, candidate peptide sequence data is generated by dividing the sequence data of a known protein into ninemer and tenmer fragments. Some embodiments feature data for a set of known peptides comprising sequence data and data regarding the affinity of the peptides for the protein.

A method for identifying epitopes is another aspect of the present teachings. In this aspect, the method comprises obtaining sequence data for candidate peptides, obtaining data for a set of known peptide epitopes that bind a MHC protein, and evaluating the affinities of candidate peptides for the MHC protein using two or more prediction methods along with the data for known peptide epitopes. Furthermore, the method comprises assigning the candidate peptides to one of two or more groups based on the evaluations of affinity, and identifying a set of candidate peptides by group assignment. In some embodiments, the epitopes are T-cell epitopes. In some embodiments, the MHC protein is a class I MHC protein. In some of those embodiments, the class I MHC protein is one of the following allotypes: A1, A2, A3, A24, or B7. Some embodiments feature quadratic programming, linear programming and profile-based methods as prediction methods. In those embodiments that use profile-based methods, methods such as iterative multiple alignment, letter frequencies, and position dependencies reflected by χ² tests can be used. Particular embodiments use quadratic programming as one of the prediction methods. In some of these embodiments, a data reduction method (e.g, a Feature-space reduced method), such as a BIMAS-like or AA-properties method are used. In certain embodiments, the evaluations of affinity comprise the assignment of ranks to candidate and known peptides based on their relative affinities to the MHC protein.

Another embodiment of the present teachings is a computer system for predicting the affinity of candidate peptides for a protein that comprises a database containing data for sets of known peptides, a database containing sequence information for the candidate peptides, a processor capable of evaluating peptides by multiple methods and a database for storing evaluations. In some embodiments, the system includes programming for the execution of the multiple methods of analyzing the candidate peptides. In some embodiments, the data for sets of known peptides comprises sequence data and data regarding the affinity of the known peptides for a protein. In certain embodiments, the sets of known peptide contain peptides that are epitopes with affinity for or that are recognized by immune system proteins.

### BRIEF DESCRIPTION OF THE DRAWINGS

FIG. 1 represent a general overview of the present teachings. Data regarding known peptide-protein interactions is combined with algorithmic methods of binding prediction and sequences of candidate peptides to create predictions of binding capacity for the candidate peptides. These predictions can be used to select candidate peptides for further study or uses.

FIG. 2 represents a strategy for binding level prediction using the present teachings. Information about known epitopes is used with algorithms and sequences of candidate peptides to classify the candidate peptides based on predictions of their level of binding strength. The classifications assign a range of absolute binding strengths to candidate peptides.

FIG. 3 represents the epitope prediction pipeline. Input protein sequences are processed to yield a peptide database. Successive prediction methods are used with the peptide elements of the database, yielding a final ranked and annotated list of peptides for each allele.

### DETAILED DESCRIPTION OF THE PRESENT TEACHINGS

### I. Introduction

The data provided by the completion of the human genome project has the potential to revolutionize the design of medical treatments. Knowledge of the complete repertoire of human proteins should allow researchers to decrease the amount of trial and error experimentation required for the discovery of new biologically active agents. For example, in the field of computational immunovaccinology, or computer aided vaccine design, theoretical bioinformaticists and vaccine experimentalists are attempting to use computational methods to rapidly and inexpensively screen among billions of peptides to identify peptides with therapeutic potential. Information derived from the sequencing of the human genome could be used in the design of new vaccines. Handling the enormous amounts of data that the sequencing of the human genome has provided, however, will require new methods for manipulating and processing data.

Vaccines are designed to create or promote an immune response by the body; potential targets of such a response include foreign organisms, virally infected cells and tumor cells. The immune response provoked by a vaccine can be intended to treat a current disease or protect against future infection and, potentially, future malignancy. In order for the immune system to detect cells infected with viruses or those cells producing aberrant proteins, a sign of malignancy, the immune system must be able to survey the production of proteins within individual cells of the body. All cells of the body present a sample of the proteins currently being produced by the cell to the extracellular compartment. Peptides derived from proteins being synthesized by a cell are presented at the cell surface as antigen epitopes bound to MHC class I transmembrane proteins. Recognition of non-self antigens bound to class I MHC proteins by cytotoxic T cells occurs when the presented epitope binds with the T cell receptor. This recognition of a immunoreactive antigen epitope by the cytotoxic T cell leads to the destruction of the cell presenting the non-self epitope.

The peptides presented by the MHC class I protein are not a random sample of protein degradation products. At all steps in the generation and selection of these peptides, constraints are made on the length and sequence of the peptides. Such constraints occur during the breakdown of proteins into peptides and in the transport of peptides into the endoplasmic reticulum, where they interact with the MHC class I proteins before being presented on the cell surface. However, incorporation of peptides into the MHC complex appears to be the primary point at which sequence specificity constrains the selection of peptides for presentation. Thus this step is also the primary point for determining which antigens are presented for recognition. MHC proteins are membrane-bound complexes of two proteins, the alpha chain and the beta chain, that are presented on the surfaces of most cell types. In class I molecules, only the alpha chain is involved in peptide binding. The alpha chain forms a narrow groove between two alpha helices that are packed next to one another on top of a beta sheet. Antigenic peptides bind to this groove where they are presented by the cell and can be recognized by the T cell receptor. (For a thorough description of the structure and function of the MHC complex, see, for example, Janeway et al. (1999)).

The binding mechanism of the class I MHC complex was revealed by the solution of the crystal structure of MHC with a bound peptide (Madden et al., 1992). The MHC class I complex presents short peptides in a defined binding groove. The binding groove generally accommodates peptides of 9-10 amino acids, although significantly longer epitopes have been found. In most cases, peptides appear to be bound to the binding groove at both N- and C-termini (Bouvier and Wiley, 1994), although peptides can extend beyond the groove (Collins et al., 1993). Peptides are also bound to the MHC complex through “anchor sites,” pockets in the MHC complex that accommodate some peptide side chains. These pockets also appear to be a major source of the peptide specificity of the MHC complex. On class I MHC proteins, the anchors are located near the ends of the peptides (typically the second and last positions), allowing peptides to bulge out in the intervening region; the bulge can be of variable size, allowing additional flexibility in peptide lengths (Guo et al., 1992).

The MHC genes are highly variable among human populations, with different alleles conferring different peptide specificities. The peptide specificity of the MHC is determined by the HLA alleles that encode the MHC proteins. Hundreds of distinct HLA alleles have been observed, with variation occurring predominantly at those sites involved in peptide binding. These alleles have been organized into classes of similar alleles with nearly identical specificities, which are known as HLA supertypes or allotypes. Table 1 in the Background section shows the anchor specificities for ninemer peptides binding to the five major MHC class I allotypes: A1, A2, A3, A24, and B7.

The first attempts to understand the constraints on the sequences of peptide antigens presented by MHC class I proteins were reported in studies by Falk, Hunt and colleagues (Falk et al., Nature 1991; Hunt et al., Science 1992). In these studies, the isolation of endogenous peptides from five murine and human class I molecules led to the hypothesis that the endogenous peptide repertoire is largely determined by a limited set of amino acids, thereafter called anchor residues, at certain positions in the peptides. Moreover, the exact location of anchor residues within peptides binding to particular class I molecules was conjectured to be a direct reflection of the HLA pocket accessibility and composition. In studies by Falk and DiBrino, motif patterns emerged that featured the amino acid Leu in position 2 (P2) of epitope peptides and amino acids Tyr or Lys at P9. In addition to these primary anchors, secondary (auxiliary) anchors, such as Phe at P3, were identified as contributors to the binding affinity of the ninemers tested. Further studies (Ruppert et al., 1993) confirmed that primary anchors are necessary, but not sufficient, for high affinity binding, and demonstrated prominent roles for several other positions. It was alos discovered that the use of extended motifs which take into account secondary anchors increased the predictability of HLA-A0201 binding epitopes from 30% to 70%. The biological bases of these observations were derived from the fact that the locations of the auxiliary residues within the peptides corresponded to secondary pockets previously demonstrated by X-ray crystallography.

Most of these observations were obtained experimentally, with limited resources and scope. The first approach to large-scale in silico epitope prediction based on anchor identification was taken by Rammensee et al. (Rammensee et al., 1999). It produced an algorithm for predicting epitopes from protein sequences, and a database (SYFPEITHI; http://syfpeithi.bmi-heidelberg.com.) of experimentally identified and published motifs, both publicly accessible through a web interface. In the algorithm, each amino acid in a candidate peptide is assigned a score that depends on the type of residue, the type of position (anchor, auxiliary), and the frequency of that amino acid at that position in the database of published motifs. The overall peptide score is the sum of the individual amino-acid scores. Ultimately, high-scoring peptides predicted as immunogenic need to be experimentally validated with binding assays and in vivo and in vitro T-cell assays before becoming good candidates for cancer vaccines.

More recently, EpiMer (De Groot et al, 2001) emerged as a pattern-matching prediction algorithm based on the same principles of anchor detection. It uses a library of published anchor-based MHC binding motifs for class I and class II HLA alleles to search for regions containing potential epitopes. However, instead of limiting its scope to detecting motifs for one allele type, it attempts to identify MHC ligands containing patterns that allow binding to more than one type of MHC molecule. Such peptides are referred to as ‘promiscuous’, and are an effective way of surmounting the hurdles of genetic variation when selecting peptides that would stimulate a protective immune response.

While anchor specificities largely describe the peptide constraints on MHC binding, they are far from a complete description of the binding specificities of individual alleles. Other positions, such as secondary anchors, appear to play a lesser role in determining allele-specific binding preferences. In addition, the assumption that binding residues exert contributions independent of one another, while apparently a good approximation, is imperfect.

To improve upon peptide epitope prediction using anchor specificities, matrix-based methods for MHC binding were introduced in Parker et al. (1993). The matrix-based methods may be seen as a direct extension of the anchor based ones. In these methods, it is assumed that the binding of an epitope to the MHC allele cannot be solely explained by the binding of one or more anchor amino acids, but rather the whole peptide needs to be considered.

At the heart of the matrix-based methods is the assumption that the binding of each individual amino acid of an epitope peptide to the MHC molecule is independent of the binding of the other amino acids in the peptide. This assumption is known as the independent binding strength assumption (IBS). Each amino acid in the peptide contributes independently to the binding of the peptide to the MHC molecule. Thus, each amino acid in each position can be considered to have an associated binding constant to the MHC molecule and the binding of the peptide to be a conjugate of those individual contributions.

Matrix-based methods can be demonstrated with a simple mathematical model. The half-life of binding (or IC-50 value) of a peptide ρ=a₁a₂a₃a₄a₅a₆a₇a₈a₉ can be modeled as

$$B ( ρ ) = {\prod }_{i = 1}^{9} B ( {a}_{i} ) \cdot c .$$

By taking logarithms of the previous equation, or alternatively looking at the binding energy, the equation can be written as

$$log ( B ( p ) ) = E ( p ) = {\sum }_{i = 1}^{9} log ( B ( {a}_{i} ) ) + {c}_{2} .$$

To predict the binding constant of all peptides, all that is needed is the binding strength of the 20 different amino acids in 9 different positions, a total of 180 values. Hence, the information in a matrix measuring 20 positions by 9 positions can generate a prediction of the binding strength of a peptide for a class I MHC protein.

Experimentally, these binding constants can be predicted by doing single base pair substitution experiments: The IC-50 value for the binding of one template peptide to the MHC allele of interest is measured. Then, given number epitopes 9·19=171 different peptides are generated, all of which differ from the original template by a single base pair. For each of these peptides, the IC-50 value is measured. The binding constant associated with the amino acid that was substituted is then the difference between the logarithm of the IC-50 value of the template peptide and the logarithm of the IC-50 value of the new peptide.

An alternative method applied by Parker et al. (1993) to derive matrix values which can make use of a more diverse collection of data is to perform a linear regression. The basic version of the approach can be formulated mathematically as follows. Given a set of ninmer peptides and a measurement of their binding strength to the MHC, the set of peptides is mapped to 9*20=180 dimensional space. One variable is constructed for each amino-acid in each position. A peptide can be mapped to a point, in 180 dimensional space, that has value 1 if the peptide has the given amino acid in the given position and 0 elsewhere. This mapping creates a set of points (peptides), xᵢ, and with associated values, bᵢ (binding strength). Under the IBS assumption, the binding strength is a linear function of the points in this space. The goal is to determine the function that best fits the data. This can be solved using linear regression; for the set of data-points, in order to minimize the difference between the predicted binding strength and the actual binding strength.

There can be some difficulties in using the linear regression approach to create a peptide binding model. The number of space dimensions can be large (180) and the number of good data points is often small. Furthermore, the data points are not usually evenly sampled in the space. This may cause overfitting predictions that are based on noise in the data and not the physical properties that researchers are attempting to model. It would therefore be advantageous in some circumstances to decrease the number of space dimensions.

One way to achieve a reduction in the number of space dimensions is to assume that similarly structured amino acids can be expected to have similar binding strengths. The model can group the amino acids into classes and require that the amino acids within each class have the same binding strength.

Parker et al. (1993) looked at the binding of a peptide to the MHC and inferred which properties of the amino acids are the most important in the binding. Two types of predictions were made regarding the properties of the amino acids in the binding peptides. Firstly, some amino acids in some positions are considered to not affect the binding of the peptide to the MHC. Secondly, certain groups of amino acids are considered to have the same effect on binding. In this way, Parker et al. (1993) reduce the space dimensions for the binding of peptides to the MHC A2 to 46 from 180.

An alternative to inferring binding strengths from binding strength data of known sequences is the estimation of binding strengths from predicted structures. Structure-based inference could be expected to eliminate some of the data-dependence of sequence-based methods. They also might be able to give greater accuracy, especially for peptides with sequence characteristics poorly represented in available epitope databases.

Rognan et al. (1994) has used molecular dynamics methods to study the nature of the interaction of peptides and MHC proteins at an atomic level. In later work, Rognan et al. (1999) built a structure-based prediction method for MHC binding strength calculations using detailed atomic-scale energy calculations, which was centered around the concept of a force field specifically designed for MHC binding strength prediction. The Rognan method depends on crystallographic structures that are available for several MHC complexes with bound peptides. Given an allele for which no structure exists, a predicted structure for that allele is developed using homology modeling of similar alleles of known structure. A target peptide is then modeled into the binding groove of the allele structure, minimizing energy as predicted by the FRESNO energy function, which Rognan et al. developed specifically for predicting MHC binding energies. Later work (Logean et al., 2001) showed that the custom energy function to be significantly better than more general energy functions at predicting MHC binding energies. The final minimum energy score achievable by the target peptide in the allele structure is the estimated affinity of the peptide.

One structure-based method was developed by Altuvia et al. (1997) and Schueler-Furman et al. (2001). This method uses solved crystal structures of MHC-peptide complexes as a template, then applies a threading method to model new peptides onto known peptides. The approach uses pairwise potentials between residues pairs, one residue on the MHC protein and the other on the peptide, that exist in close proximity to one another in the three-dimensional structure of the peptide-bound MHC protein. These potentials are obtained from solved structures of peptide-bound MHC molecules and provide a relatively simple computational method for estimating the binding energies of new peptides.

Both methods appear to have a higher accuracy compared to sequence-based methods, although they also have significant drawbacks. Rognan et al. determined the standard error on a set of test proteins to be approximately 3.49 kJ/mol. Their testing was performed on a data set that was also used to optimize the energy function, however, and may not be indicative of performance on independent data. Both methods would be expected to perform poorly on any peptides that bind in ways that are not represented in solved crystal structures. The most significant drawback of the structure-based methods, though is that they are far more computationally costly than sequence-based methods and would therefore be more difficult to use for screening large numbers of potential epitopes. Pairwise potentials can be expected to be considerably less costly than more realistic force-fields, although they can also be expected to have lower accuracy.

An approach complementary to predicting peptides with good binding and immunogenic properties is to generate peptide analogs, by modifying a given epitope, that are more potent and immunogenic than the wild-type sequence. In earlier studies, peptide analogs with increased binding affinity were produced by modifying the residues at the anchor positions (Valmori et al., 1998; Bakker et al., 1997). More recently, non-anchor positions have been targeted as well. While single substitutions analogs of an antigenic peptide at non-anchor positions have in general a null or detrimental effect on T-cell activation, some analogs, so called heteroclitic, have unexpectedly increased potency. Such analogs may provide considerable benefit in vaccine development, as they induce stronger T-cell responses than the original epitope, and increased affinity of the epitope/pMHC complex for the TCR molecule. In a recent report, heteroclitic analogs were obtained computationally by conservative or semiconservative substitutions at odd-numbered positions in the middle of the peptide, and validated by in vitro testing of the cytotoxic T-lymphocyte (CTL) response (Tangri et al., 2001). Peptide analogs obtained by conservative and semiconservative substitutions at positions P3 and P5 were then tested and validated as heteroclitic by measuring the CTL response.

### II. Methods

Several embodiments of the present teachings include some or all of three different classes of algorithms for classifying peptides as epitopes. In an additional embodiment, algorithms are integrated into a computational pipeline for high-throughput analysis of protein sequences. Some of the methods presently taught use algorithms to learn motifs for the epitopes and construct matrices for the sets of amino acids in different positions. These matrices can be used to identify novel candidate peptides that represent a desired level of binding to a protein.

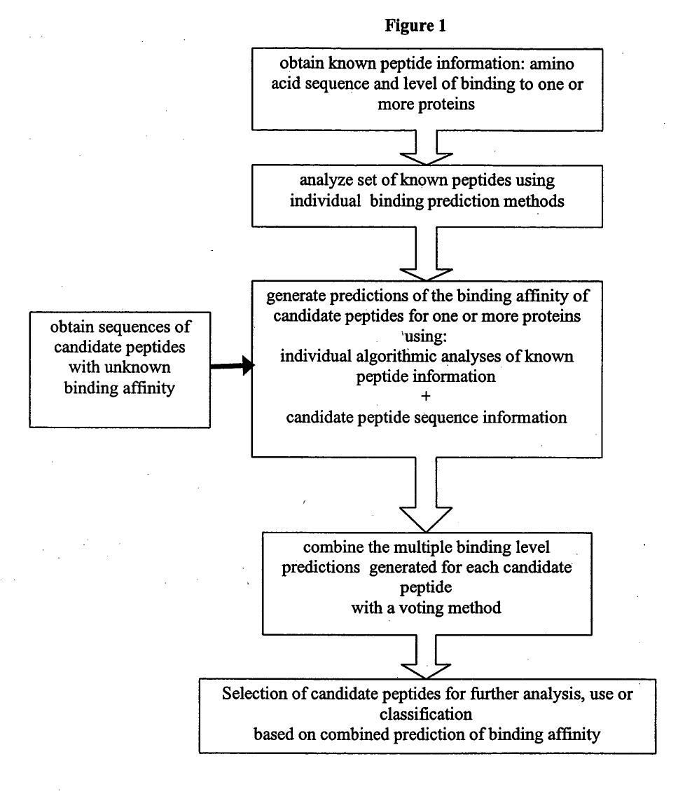

A flowchart outlining a general strategy for predicting the binding strength of peptides using some of the present teachings is presented in FIG. 1. Information regarding the sequence and binding affinity of peptides whose interaction with a protein has been observed is used with computational methods of the present teachings. The binding strength of candidate peptides can then be predicted given the sequence of the peptides by combining the estimations of binding affinity produced by the methods of the present teachings. Candidate peptides can be selected from the group based upon these combined predictions of binding strength.

The quadratic programming technique discussed below is one embodiment of the teachings. Methods based on this technique can take into account more subtle ordering and dependencies between amino acid binding contributions than matrix-based techniques and can be used with qualitative or quantitative data. Another class of algorithm, linear programming techniques, explores the linearity of amino acid contributions and can be used to minimize the overall number of epitopes under consideration. A third type of algorithm technique, the profile-based methods, assumes that there are more than one motif describing the set of epitopes for any given allele. Profile-based methods can propose ways of clustering the known epitopes to identify motif classes. In an additional embodiment of the teachings, a voting algorithm is used to integrate the results from the various prediction methods to produce a more accurate final binding score. This final binding score can be further used to classify the peptides into categories according to their binding level (such as, for example, high, medium, low, and non-binding).

A. Quadratic Programming

One embodiment of the present teachings features a quadratic programming approach to estimate the binding affinities of candidate peptides for a protein. Quadratic programs (QPs) are used to maximize or minimize a quadratic objective function that is constrained by a set of linear constraints. QPs can be solved using a variety of standard software packages, including Matlab and CPLEX.

A primary advantage of the method over previously described methods is that it allows us to incorporate information gained from sources alternate to binding half-life (IC-50 value) measurements. In general, for experiments designed to measure the binding of peptides to proteins quantitatively, it can be difficult to calibrate the measurements of binding between different experiments. This is due in part to the incompatibility of the measurements performed by different groups of experimenters or with different experimental protocols. In some embodiments, quadratic programs may be seen as the following quadratic minimization problem:

$${min}_{w , c} {\sum }_{i}^{} {( {x}_{i}^{T} w - c - {b}_{i} )}^{2}$$

In this particular embodiment, the data input is a set of peptides, xᵢ, and a measurement of their binding strength, bᵢ. The variable w is a vector of predicted binding constants and c is an offset from zero. The term xᵢ^{T}w-c is thus the predicted binding constant of a peptide, and xᵢw-c-bᵢ is the difference between the predicted and measured binding strengths, attributable to experimental or modeling error. In some embodiments of the present teachings, the goal of this algorithmic approach is to find a vector of weights w and a constant c that minimize the sum of the distances to the set of observations.

Quadratic programming can help overcome a primary disadvantage of the linear regression approach, which is that data is not always given in terms of binding strength. The only available information may be whether the peptide is an epitope or not. In some embodiments of the teachings, this data can be added to the model by adding a constraint. It is required that the model assigns to epitopes, i.e. peptides that bind to the protein, a binding strength greater than the minimum binding strength of known epitopes. Non-epitopes are assigned binding strength less than the minimum binding strength of known epitopes. In addition to whether a peptide is an epitope or not, it may also be given whether a peptide is a High, Low or Medium binder, in certain data sets. In some embodiments, this information can also be incorporated in the model by requiring, for example, that the high binders have a higher binding strength than the low and medium binders.

One embodiment of the present teachings comprises an alternate quadratic program that can be used with categorical binding data from peptides. Let x_{H}, x_{M}, x_{L} be high, medium and low binders, let xₑ be epitopes, xₙₑ be non-epitopes, and let IC₅₀^{min} be the minimum binding strength of an epitope. In this case, the quadratic program can be modeled as follows:

$${min}_{w , c} {\sum }_{i}^{} {( {x}_{i}^{T} w - c - {b}_{i} )}^{2}$$

$$s . t . {x}_{{H}_{i}}^{T} w \geq {x}_{{M}_{j}}^{T} w ∀ i , j , \\ {x}_{{H}_{i}}^{T} w \geq {x}_{{L}_{j}}^{T} w ∀ i , j , \\ {x}_{{M}_{i}}^{T} w \geq {x}_{{L}_{j}}^{T} w ∀ i , j , \\ {x}_{{e}_{i}}^{T} w \geq {IC}_{min}^{50} , {x}_{{ne}_{j}}^{T} , w \leq {IC}_{min}^{50}$$

This quadratic program may have a set of constraints that are not feasible, i.e. no set of parameters w and c will satisfy all the constraints. This could be due to inaccuracy in the experiments or because the independent binding strength assumption may be incorrect to some degree with regard to the particular protein in question. To overcome these obstacles in the model, in some embodiments, violations of the constraints are penalized and the set of parameters that are in the least violation to the set of constraints is determined. With the incorporation of these penalties, the model is then as follows:

$${min}_{w , c} {\sum }_{i}^{} {( {x}_{i}^{T} w - c - {b}_{i} )}^{2} + {\sum }_{i , j}^{} {e}_{{H}_{i} {M}_{j}}^{2} + {\sum }_{i , j}^{} {e}_{{H}_{i} {L}_{j}}^{2} + {\sum }_{i , j}^{} {e}_{{M}_{i} {L}_{j}}^{2} + {\sum }_{i , j}^{} {e}_{{e}_{i}}^{2} {\sum }_{i , j}^{} {e}_{{ne}_{i}}^{2}$$

$$s . t . ( {x}_{{H}_{i}}^{T} - {x}_{{M}_{j}}^{T} ) w + {e}_{{H}_{i} {M}_{j}} \geq 0 ∀ i , j \\ ( {x}_{{H}_{i}}^{T} - {x}_{{L}_{j}}^{T} ) w + {e}_{{H}_{i} {L}_{j}} \geq 0 ∀ i , j \\ ( {x}_{{M}_{i}}^{T} - {x}_{{L}_{j}}^{T} ) w + {e}_{{M}_{i} {L}_{j}} \geq 0 ∀ i , j$$

$${x}_{{e}_{i}}^{T} w - {IC}_{min}^{50} + {e}_{{e}_{i}} \geq 0 ∀ i$$

$${IC}_{min}^{50} - {x}_{{ne}_{i}}^{T} w + {e}_{{ne}_{i}} \geq 0 ∀ i$$

Data Reduction

Some embodiments of the present teachings use data reduction techniques to simplify calculations and streamline the prediction process. Several approaches to reducing space dimensionality are used individually or in combination with one another by some embodiments, which can lead to differing matrices and prediction methods.

One method of data reduction used in some embodiments is known as the ‘BIMAS-like’ method. In the ‘BIMAS-like’ method, data reduction techniques are used that are similar to the techniques applied by Parker et al. (1993) to derive their matrices. To achieve data reduction with this method, relations were derived regarding which amino acids are irrelevant for binding and which pairs of amino acids have an equivalent effect on binding. For example, in the case of the A2 allele, Asp and Glu are assumed equivalent for binding in the first position. The matrix values can then be obtained for the data reduction model by further constraining the model to respect these relations.

Other embodiments used a method of data reduction called ‘AA-properties’. In this method, the feature space is reduced by using properties of the amino acids. For example, hydrophobicity and size can be used to reduce the size of the space. Thus, a ninemer peptide can be mapped into a 9*2=18 dimensional space (nine residues*two properties per residue). Each amino acid is mapped to a point in two dimensions, first as a measurement of its hydrophobicity, and second as a measurement of its size. Various indices reported and published in the AAindex repository (Kawashima et al., 1999) can be used to reduce the data. In some embodiments, a subset of parameters that best fit the data are chosen from these indices, using a feature selection heuristic.

B. Linear Programming

Some embodiments of the present teachings employ linear programs (LPs). LPs maximize or minimize a linear objective function, constrained by a set of linear constraints, and can be solved using a variety of standard software packages, including Matlab and CPLEX.

The development of the linear programming formulation was motivated by the fact that input data often has more positive examples (epitopes) than negative ones (non-epitopes). A goal of some embodiments is to find a finction that assumes large values for epitopes, but not for non-epitopes, so as to distinguish between epitopes and non-epitopes. In some embodiments, artificial ‘binding strengths’ are constructed for each amino acid in each position. In this formulation, epitopes are required to have a high binding strength, but the sum of the binding strength of those peptides considered epitopes is kept at a minimum. Minimizing the sum of the binding strengths is considered a proxy for minimizing the number of peptides considered epitopes. Therefore, this method attempts to minimize the number of peptides classified as epitopes while requiring known epitopes to be classified as such. In order to avoid artificially low objective values, all binding strengths are required to be positive.

In particular embodiments, artificial ‘binding strengths’ for each amino acid in each position are constructed and each is required to be positive. The sum of the binding strengths for each epitope is required to be greater than 1, an arbitrarily chosen constant. The sum of the ‘binding strengths’ is then minimized. Let bₐₚ be the artificial binding strength of amino acid a in position p, eᵢ be the i-th epitope, eᵢ^{p} be the amino acid in position p of epitope eᵢ. The following linear program is formulated and solved for bₐₚ.

$$min {\sum }_{{aa}^{′} sa}^{} {\sum }_{pos . p}^{} {b}_{ap}$$

$$s . t . {\sum }_{pos . p}^{} {b}_{{e}_{i}^{p} p} \geq 1 ∀ i$$

$${b}_{ap} \geq 0 ∀ a , p$$

C. Profile-based Methods

Profiles were introduced in the bioinformatics literature in the context of motif detection in DNA and protein sequences (Gribskov et al., 1987; Thompson et al., 1994; Bairoch et al., 1996). In particular, they have been used extensively for identifying cis-regulatory elements in DNA sequences (Stormo 2000). Epitopes and MHC binding peptides can contain allele-specific sequence motifs, where the sequence composition of the various positions, and the degree of variation at these positions, are dictated by the specificity of the secondary structure pocket of the MHC. For instance, anchor positions, which are more influential than the others in securing the MHC molecule-peptide complex, have a very narrow range of allowed amino acids, directly reflecting their increased specificity.

A sequence profile, or simply a profile, is a representation of a set of aligned sequences as a table of letter frequencies or absolute counts per each column in the alignment, together with a position-specific weight matrix derived from the alignment data. An example is provided in Table 2 below, in this case a profile of nucleic acid sequences. This encoding captures both the variability of symbols at the various alignment (motif) positions, and a characterization, or measure, for their variation.

TABLE 2
Profile representation of a multiple aligmnent of
six sequences. The element N(X, k) in the
associated table is the number of occurrences of
letter X in column k.

| Alignment: | Table of letter frequencies-N: |
|---|---|
| ATACCTTAT | 1 2 3 4 5 6 7 8 9 |
| TTACTTAAT | A \|3 0 6 1 1 3 4 5 0 |
| TTACAATAT | C \|0 0 0 5 1 0 0 0 0 |
| ATACTAAAT | G \|0 0 0 0 1 0 0 1 0 |
| ATACTAAGT | T \|3 6 0 0 3 3 2 0 6 |
| TTAAGTAAT | |

In previous prediction studies, the set of epitopes for each type of HLA molecule was assumed characterized by only one motif class. In some embodiments of the present teachings, the existence of multiple sequence motifs within the set of epitopes for any given allele is hypothesized. Some embodiments develop methods to divide the epitopes into several distinct motifs, each described by a sequence profile object. This intra-allele differentiation of sequence profiles allows some embodiments of the present teachings to more precisely distinguish between candidate peptides with different capacities for protein binding.

In some embodiments of the present teachings, profile-based approaches to epitope prediction rely on some or all of three principles: dimensionality reduction, multiple intra-allelic motifs, and anchor selection.

The first principle, dimensionality reduction, is based on the observation that certain amino acids have similar functions at a given position, explained by the compatibility of their side chains with the secondary structure pocket (Parker et al., 1994b), and therefore can be deemed functionally equivalent. For instance, Leu and Met are both hydrophobic and preferred at the P2 anchor position in HLA-A2 ligands. Using the priniciple of data reduction with profile-based methods, a simple equivalence scheme is selected in some embodiments of the present teachings that divides the amino acids in four classes (hydrophobic=H, polar=P, charged=C and glycine=G), based on the reference biochemical classification of (Branden and Tooze, 1991). The hydrophobic class consists of the amino acids Ala, Val, Phe, Pro, Met, Ile and Leu; the polar class groups Ser, Thr, Tyr, His, Cys, Asn, Gln, and Trp; the charged group contains Asp, Glu, Lys and Arg; and Gly is alone in the glycine group. Such a partitioning assumes a uniform substitution model for amino acids across the peptide and across the range of HLA molecules. The biochemical signature of a given peptide or protein sequence is then the direct ‘translation’ of the peptide into this four-letter alphabet; for instance, the biochemical signature for YLLPCITEV is PHHHCHPCH. In these embodiments, profile-based methods of peptide binding are then performed on the biochemical signatures of the peptides in lieu of the peptides themselves.

Some embodiments incorporate a second principle, multiple intra-allelic motifs. This principle is based on the hypothesis that epitopes for any given HLA molecule can be classified in one or more classes of motifs. As an example of this principle, evidence of position dependencies can be observed among the epitopes for a given allele in Table 3 below, which points to groups of sequences with distinct features. The top line shows the distribution of randomized amino acids reduced to the terms of biochemical signatures, i.e. 20% of amino acids fall into the charged catagory. Line 2 shows the distribution of all of the amino acids found in the third position of the epitope peptides of the A2 allele epitope set. Lines 3 and 4 add constraints, where the biochemical signature of the residue at the fifth position is limited to two of the four signature classes.

TABLE 3
Distributions of amino acid types at P3 in the sequences of HLA-A2
ligands and epitopes: (1) in a uniform distribution model; (2) in the
original data set; (3) in the subset of sequences that contain a
hydrophobic (H) or polar (P) residue at P5; (4) in the subset of
sequences with a charged (C) or glycine (G) residue at P5..

%

| | P | C | G | H |
|---|---|---|---|---|
| (1) Uniform distribution model | 40 | 20 | 5 | 35 |
| (2) Distribution of residue types at P3 | 32.2 | 13.4 | 11.4 | 43.0 |
| (3) (PH)₅-restricted P5 | 38.2 | 11.8 | 5.5 | 44.5 |
| (4) (GC)₅-restricted P5 | 15.4 | 17.9 | 28.2 | 38.5 |

Note that in Table 3, the amino acid type distribution in the sample set is not random, by comparing lines (1) and (2). Also, that significantly different letter profiles are observed for the P5-PH restricted and P5-CG restricted sequence subsets. Lastly, that C or G type residues at P5 strongly favor glycine (G), and restrict polar (P) amino acids at P3. These observations are based on a data set of 149 samples. The table illustrates the interdependencies of the identities of residues at various positions to one another and the existence of distinguishable motifs for MHC protein alleles.

Some embodiments use a third principle, anchor selection, which takes into account the fact that anchor positions have a lower degree of variation than the rest of the positions in the peptide. This aspect of peptide sequence constraint can be overlooked in other principles of profile-based methods. For instance, a ‘hydrophobic’ label at P2 of a predicted HLA-A2 epitope can encode any of the following seven amino acids: Ala, Val, Phe, Pro, Met, Ile, and Leu; all of these will produce the same score. In reality, only five (Ile, Leu, Val, Met and Ala) have been observed in practice at the P2 position in HLA-A2 epitopes, and with widely varying incidence rates. In some embodiments, in order to compensate for this loss of specificity, a more differentiated scoring method for the identity of amino acids is used at known anchor positions.

As used in certain embodiments of the present teachings, a profile is a compact representation of a motif class as an alignment of (usually) fixed-size sequences, together with a position-specific weight matrix derived from the letter frequencies for each alignment column. There is a matrix element for all possible bases at every position in the profile. The score for a candidate sequence is then the sum of matrix values for the individual ‘letters’ in that sequence. The higher the score, the better the candidate sequence conforms to the motif pattern and the more likely it is that it belongs to that motif class.

In some embodiments of the present teachings, profiles were constructed using the alignment of biochemical signatures of experimentally validated epitopes and ligands. The

score of a candidate peptide was computed as the sum of nucleotide weights (Gelfand et al., 2001):

$$Score ( a1 a2 … ak ) = W ( a1 , 1 ) + W ( a2 , 2 ) + … + W ( ak , k ) , \\ with$$

$$W ( b , i ) = log ( N ( b , i ) + .5 ] - .25 {* Sum}_{a = P , H , C , G} log [ N ( a , i ) + .5 ] , b ( \{ P , H , C , G \}$$

where N(a,i) is the number of occurrences of amino acid type a in column i. Since the primary goal in these embodiments was to rank the candidate peptides by score, rather than select a subset to predict as epitopes, a score threshold was not necessary. However, lower bounds for threshold can be obtained under the constraints of conservative extension. Intuitively, sequences in the profile should score higher than random sequences.

One embodiment of the present teachings features a profile-based method for predicting epitopes in a given protein sequence. A comprehensive database of all overlapping peptides 9 or 10 residues long is generated. For a chosen allele, each candidate peptide of length 9 (ninemer) is aligned with each of the profiles for this HLA allele, and a score is generated. The final peptide score is either the maximum of individual profile scores (the MAX method), or the score produced by the specific profile whose sequence composition matches that of the candidate peptide (the MDD method). The MDD method is typically used with the LetFq and χ² profiles, described below, which divide the set of all possible sequences into disjoint subgroups based on the amino acid types inhabiting selected motif positions, guided by a decision tree. For peptides that are 10 residues long, the final score is computed by taking the average of scores for the seven ninemers obtained by removing exactly one of the non-anchor residues, at positions P3, P4, P5, P6, P7, P8 and P9.

Clustering Heuristics

As illustrated above in Table 3, there can be interdependencies between the identities of residues at various positions within a binding peptide and multiple distinguishable motifs within a set of peptide epitopes for a MHC allele. Some embodiments of the teachings use clustering heuristics to develop binding motifs for peptides that bind a protein.

In certain embodiments, each set of epitopes and ligands is divided into clusters representing different classes of motifs, using one of the three following heuristics: iterative multiple alignment (Aln), letter frequencies (LetFq), and position dependencies reflected by χ² tests (Ki2). Aln is a greedy method that gradually builds a profile by adding a new sequence to an existing cluster, until a stop condition is encountered. The procedure is then repeated with the unassigned sequences. In contrast, LetFq and Ki2 use a divide et impera approach, which recursively splits the current sequence cluster into two disjoint subclusters, according to the sequence letter at a chosen profile position, and repeats the procedure for the two subclusters thus formed. Such strategy is best represented as a binary decision tree.

For some embodiments that use profile-based methods, an ideal partition of the epitopes into clusters would maximize the intra-cluster cohesion and minimize the inter-cluster cohesion, where the degree of cohesion can be measured by sequence similarity. While this optimization problem can be hard to capture in a mathematical framework, and the optimal solution can be therefore unknown, all of the three heuristics produced approximations that work well in practice. Moreover, some embodiments use the concept of information content as a measure of alignment quality (Stormo and Hartzell, 1989; Hertz et al., 1990). With this technique, it can be shown that the quality of the profiles generated at each division step is higher than that of the original. This shows that the profiles improved, and correspondingly the signals encoded in the profiles were amplified, with splitting.

As mentioned above, some embodiments employ methods of clustering via iterative multiple sequence alignment (Aln). The concept behind this method is that similar sequences will cluster around ‘seeds’. It uses a greedy strategy to start and grow a multiple sequence alignment (profile), by optimally choosing the next sequence in the multiple alignment as the one which maximizes the multiple alignment score. The publicly available CLUSTALW tool (Thompson et al., 1994) was used to produce the multiple alignments. At each iteration, sequences that do not improve the current score are removed from the pool, so as to ensure that the clusters converge towards a unique signal. The procedure stops when no sequences can be recruited. The current cluster is saved as a new profile, and the procedure is repeated with the remaining sequences and a new seed pair. The resulting profiles may include gaps, which are made explicit in the profile model. At the end of the process, some manual intervention may be necessary to redistribute the sequences and constrain the alignment of anchors.

Some embodiments of the present teachings use methods of clustering via observed letter frequencies per column (LetFq). In these approaches, clustering is based on the assumption that the presence or absence of a certain amino acid type at position i may compensate for or favor certain combinations of amino acid types in the remaining positions in the peptide. For instance, the absence of the consensus amino-acid at one anchor position may require that the favored amino acid(s) be present at the other primary or secondary anchor locations, for the peptide to retain its binding and immunogenic properties. Evidence for such effects is presented in Table 3.

In some embodiments, the LetFq method recursively selects a position i in the profile and a letter I in that column, divides the set of epitopes into subsets according to the amino acid type in column i, and repeats the procedure on the two subsets of epitopes created by the division. To determine the (i,l) pair at each step, the ratios between the frequencies of the most and second most common letters in each column are computed, and the column with the highest ratio is selected, if this value is larger than a constant k (=2). The procedure stops when no column satisfies this condition, or when the original cluster or any of the clusters created by partition contains less than L=5 sequences. By construction, this procedure produces ungapped profiles.

Some embodiments use methods of clustering that are guided by position dependencies observed with χ² statistical significance tests (Ki2 ). These methods represent another way to capture an idea presented earlier, namely that the choice of amino acid at one or more positions in the peptide may influence the distribution of amino acid types on the remaining motif positions, as seen in Table 3. In particular embodiments, to capture the dependencies between pairs of columns, and between a column and the rest of the alignment positions, χ² statistical significance tests are used. Let Ci be the consensus (majority) variable for column i (1 if the amino acid type at that position matches the consensus, 0 otherwise), and Xi the indicator variable for the same column, identifying the amino acid type at that position in terms of biochemical signature (P, H, C, G). The (χ² statistics between Ci and Xj can then be used to detect statistically significant dependencies between the distributions of amino acid types at the two positions. The χ² statistics can be computed from the 2×4 contingency table of Ci and Xj with the formula ×²i,j=Σu,v [(Ou,v-Eu,v)² /Eu,v], where Ou,v is the observed frequency for the Ci=u and Xj=v event, from the (u,v) cell in the table, and Eu,v is the expected frequency of the event. These values are interpreted from the standard χ² association tables, which list the P-values for rejecting the null hypothesis that the row variable Ci is unrelated to the column variable Xj. By construction, the profiles obtained with the Ki2 method are ungapped.

In some embodiments, with this formalism in place, a collection of disjoint clusters can be generated from an aligned set of sequences, each representing a distinct sequence motif, using the most significant dependencies between positions to guide the splitting process. Given a data set of biochemical epitope signatures, consisting of sequences of fixed equal length, a consensus amino acid type is first assigned to each position. In one example, using a data set for the HLA-A2 allele, which has a pronounced proclivity towards hydrophobic and polar residues, the consensus vector is C=[PH, PH, PH, *, PH, PH, PH, PH, H]. Then, for each pair of positions (i, j) with i≠j, the χ²i,j statistics for Ci versus Xj is computed. The selection of a column that can be used to divide the sequences into two groups, those that contain a consensus letter at that position and those that do not, is then attempted. Specifically, a column l with the largest overall association χ²(1)=Σₘ χ²1,m with the rest of the positions in the alignment is selected, such that each of the two resulting subsets contains at least 5 sequences and that χ²(1)>=K (K being a splitting constant that depends on the number of degrees of freedom for the current system). The procedure is repeated for each of the subsets, in a divide et impera fashion.

In other examples of this method, a slight variation of this procedure can be used for the rest of the HLA alleles. This produces only small differences in performance when applied to the HLA-A2 set. This deviation from the earlier procedure may be necessary to handle statistical biases in the sample data collected from the MHCPEP database that can corrupt the concept of consensus letter. Indeed, this repository contains numerous samples obtained experimentally by single residue substitutions from a seed epitope, or from the simple poly-Ala motif. For these alleles, the χ² statistic can be used to test the hypothesis of association between the ‘pseudo-consensus’ indicator Ci for column i versus the amino acid type indicator Xj at column j. For each column i, the pseudoconsensus indicator Ci varies over all possible letter combinations Ci: {P, H, C, G, PH, PC, PG}. (Combinations not listed are redundant.) The column l, if any, with the largest overall association with the rest is selected using the equation equation χ²(1)=Σₘ χ²1,m>=K(dJ), where K was chosen to correspond to a P-value less than 0.5 for the current number of degrees of freedom in the system, df Subsets with less than 20 sequences are not processed.

Additional Profile Method Approaches

Embodiments of the present teachings can generate profile models that do not depend upon the independent binding strength assumption, e.g., assuming that the contributions to binding of individual residues is independent of other residues' contributions. In the intial stages of developing the profile-based methods, several apparent pairwise dependencies were found in the nascent, emerging profiles. For instance, in the analysis of the HLA-A2 allele, it was observed that Thr at P2 was found only in combination with Val at the C-terminal. If Thr is assumed to occur at the P2 anchor position independently from the amino acid at C-terminal, approximately equal frequencies of (Thr,Val) and (Thr, Leu) should be observed, where Leu and Val are the two strong anchors for the HLA-A2 class. This assumption, of the independent occurrence model, is not valid throughout the entire set of sample data. The conditional use of an amino acid (Thr) at P2 in combination with a preferred amino acid (Val) at C-terminal therefore indicates a positional dependency in the HLA-A2 motifs. Embodiments of the present teachings permit the detection of these dependencies. By adding additional constraints to the profile-based methods described above, in particular embodiments, observed pairwise dependencies can be incorporated into the modules of peptide binding prediction.

Evaluation of the Profile Methods

Some embodiments of the present teachings incorporate methods to verify the quality of the profiles and predictions generated by these methods. To measure the quality of profiles, both before and after a cluster division, the information content of the alignment I=Σ_{k=1..9} Σ_{a=P,H,C,G} f(a,k) log [f(a,k)/0.25] (Schneider et al., 1986; Stormo and Hartzell, 1989) can be used. In all cases, the information content values of the profiles after the operation were higher than that of the original, showing that the profiles have improved. Although no score threshold was necessary to differentiate between peptides predicted as epitopes and the others, predicted as non-immunogenic, consistency checks were performed that could be used to derive sensitivity thresholds. Intuitively, sequences in the profile should score higher than random sequences. This principle is called conservative extension. For any HLA allele types and sets of allele-specific profiles, the sequences in each profile can be scanned against the profile and scores computed. In all cases, as reported in the examples below, score thresholds could be derived that were sensitive under the conservative extension tests (i.e., they rendered most, if not all, of the profile sequences as ‘epitopes’) and that also had high specificity when tested on other benchmark data sets (i.e., they produced a small number of false positive predictions). Values in the 12.0-13.0 range were found to be good thresholds for the HLA-A2 Aln set of profiles, while slightly larger values (13.0-14.0) were determined for the profile sets produced with the LetFq and Ki2 methods.

Anchor Scoring

Some embodiments include methods of scoring that take into account the increased specificity of amino acid distribution at the anchor positions versus the rest of the positions in the motif. For example, when some embodiments were used to analyze data sets for the HLA-A2 allele, the pairs of amino acids at P2 and the C-terminal of peptides were extracted from the set of 206 epitopes and ligands previously published in the literature and extracted from the SYFPEITHI database. In this example, considering the alphabet (of all possible amino acids combinations (20×20=400 letters), a 1-column profile was created containing all anchors samples extracted from the data set. The same method of scoring as discussed above (Gelfand et al., 2001) was used to score candidate peptide sequences, after first stripping all non-anchor characters. More specifically, for this 1-column profile the score of a candidate anchor is its weight:

W(b)=log(N(b)+.5)−0.25*[log(N(H)+.5)+log(N(P)+.5)+log(N(C)+.5)+log(N(G)+. 5)]

In this example, the choice of alphabet and dimensionality of the profile (one-column) correctly reflects the asymmetry in the distribution of samples. Indeed, the fact that Thr at P2 was only found in combination with Val at P9 among the A2 epitopes, and not with Leu, the other strongly favored amino acid at P9, cannot be expressed in a scoring model in which each of the amino acids in a pair contributes independently to the pair's score. In other words, this example demonstrates that granularity of the scoring at the level of pairs, rather than at the level of single amino acids, may improve the overall predictive ability of the methods.

D. Voting

In some embodiments of the present teachings, a voting or polling heuristic is used to combine the scores given by the individual methods of peptide binding strength estimation. In one version of the voting heuristic, each method is allowed to contribute to a score reflecting the likelyhood that a given peptide is an epitope or not. The methods contribute equally to the overall vote. Each method gives a vote in the range between 0 and 1, and the votes of the individual methods are summed up to give a combined score. In order to have the individual methods return a value in the range between zero and one, the scores (binding constants) returned by the individual methods are linearly scaled so that the minimum value output by the method is 0 and the maximum value is 1.

In some embodiments of the teachings, the output of this heuristic is a score that indicates the likelyhood of a peptide binding to a protein. Each method can contribute to the score, which is a combination of the inputs of the individual methods. In some embodiments of the teachings, the output is a prediction of whether the peptide is an epitope or not.

In examples shown below, for the A2 allele, five methods are used in the voting heuristic: ‘AA properties’ QP, ‘BIMAS-like’ QP, linear programming, aligrinent (Aln) profile and anchor scoring. For the other alleles that were examined, A1, A3, A24 and B7, four methods are used in the voting heuristic: Parker's method using the most recent matrices maintained at the NIH BioInformatics and Molecular Analysis Section (BIMAS) site (http://bimas.dcrt.ih.gov/molbio/hla bind/), and our linear programming, alignment profile and anchors techniques.

In other embodiments, other voting heuristics can be used. Examples of other voting heuristics include nonlinear scaling of the output of each individual method and heuristics that allow different methods to have different weights in the voting.

E. Binding Level Prediction

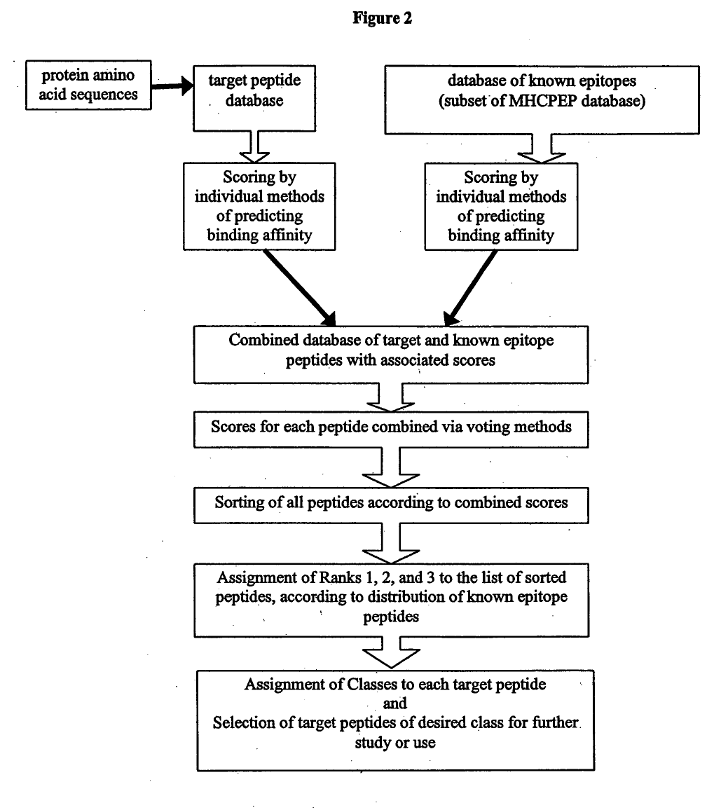

The preceding embodiments of the present teachings can be used for producing a ranked list of the relative predicted strength of potential epitopes from individual proteins. Additional embodiments of the present teachings can be used to create absolute measures of the strength of an epitope. Epitopes can be classified into four binding strength groups—high (H), moderate (M), low (L), and none (N)—following the convention used by the MHCPEP database (Brusic et al., 1997). The same definitions for the classes that are used in the MHCPEP database can be used here: high corresponds to IC₅₀<1 nM, moderate to 100nM-1 nM, low to 10 μM-100 nM, and none to >10 μM. By predicting into which class a potential epitope is likely to fall, an absolute prediction of which epitopes are likely to be effective at provoking an immune response can be derived, as opposed to the relative predictions the voting ranks would otherwise give. This method for binding level classification is based on comparison of a database of peptides to a database of known binding epitopes for any given allele under examination. In some embodiments, for the database of known epitopes for a given allele, the subset of the MHCPEP database is used, consisting of epitopes for that allele that are assigned to binding level groups within MHCPEP. In some embodiments, the overall process, given a target database of potential epitopes and a reference database of known epitopes, works as detailed below and in FIG. 2.

In some embodiments, a set of target peptides is acquired or is generated from sequence data. The generation of target peptide sequences can be accomplished by dividing the sequence of a known protein into peptide segments of nine or ten residues. A set of reference peptides, known peptide epitopes with binding level class assignments for a particular HLA allele, is chosen from the MHCPEP database. All peptides are scored using the quadratic programming, linear programming, anchor and profile-based scoring methods described previously. All of the peptides and their scores are combined into one database and the scores for each peptide are combined with a voting method. Peptides are then sorted in a list according to the combined score for each peptide, creating a list where the peptides are ranked from the highest combined score to the lowest combined score.

Some embodiments include three rank positions on the list, Rank 1, Rank 2, and Rank 3, which are assigned by scanning the list. The ranks have the following definitions. Rank 1 is the peptide rank where the number of class M reference peptides of lower rank first exceeds the number of class H reference peptides of higher rank. Rank 2 is the rank where the number of class L reference peptides of lower rank first exceeds the number of class H and M reference peptides of higher rank. Rank 3 is the rank where the last class L reference peptide is seen.

Next, the target peptides in the list are assigned to a binding level class by virtue of where they have been placed in the ranked list. Target peptides that appear on the list from the top rank to Rank 1 are assigned to class H. Target peptides that appear between rank 1 and Rank 2 are assigned to class M and those that fall between Ranks 2 and 3 are assigned to class L. Those target peptides that appear on the list between Rank 3 and the last peptide on the list are assigned to class N.

The result of this process will be an assignment of target peptides to a binding level class, where the target peptides will be grouped with reference peptides with similar levels of binding strength.

F. Epitope Prediction Pipeline

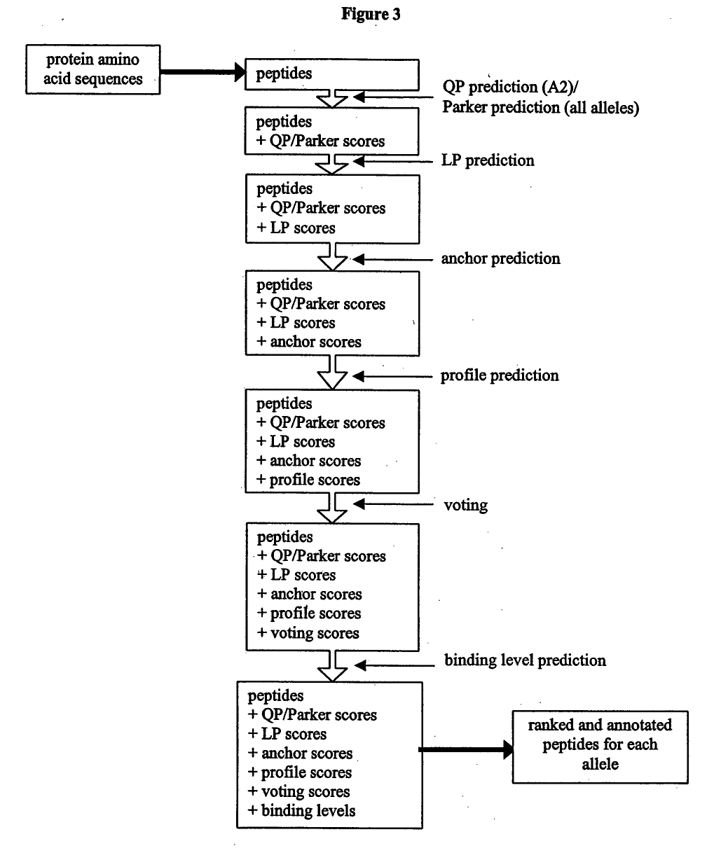

Another embodiment of the present teachings features an epitope prediction pipeline, into which the methods described above have been incorporated. In one example, a unified computational pipeline is used for producing ranked lists of candidate epitopes for each allele. In some embodiments, the overall design of the pipeline is based around a database representation of the peptide set for a given protein or set of proteins. In some embodiments, some or all of the methods described previously act sequentially on some or all peptides in the database. One possible embodiment of this pipeline is given in FIG. 3, described below.

In some embodiments, a protein under examination is first fragmented to produce all overlapping ninemer and tenmer peptides. The linear programming, anchor, and the Aln and Ki2 profile methods can be run on each peptide in the database for each of the five HLA alleles examined. In addition, the quadratic programming method and the LetFq profile method can be used for HLA-A2, and the Parker method can be used for HLA-A1, HLA-A3, HLA-A24, and HLA-B7. The voting method can then be run for each allele, producing a ranking of all peptides in the database for each allele. In addition, for each allele, the binding level prediction method can be used to assign H, M, L, and N tags to peptides. In some embodiments, the final results are presented as a database of all peptides annotated with rankings and binding level tags, as well as with the intermediate scores produced by the individual methods. See Example 1 below for additional information and results obtained using an embodiment of the present teachings.

G. Analogs

Some embodiments of the present teachings feature methods for generating analog peptides to moderate binders that are predicted to provoke an immune response that is stronger than the response generated by the wild-type peptide itself. Given a peptide that is predicted to be a moderate MHC binder, the goal of some embodiments is to find a peptide that binds more strongly to the MHC but is recognized by the same T-cell receptors as the wild-type.

In some embodiments, a simple anchor-based strategy is used. Information from MHC-peptide crystal structures indicates that the primary anchor residues are buried in the MHC peptide binding groove. The location of these primary anchor residues suggests that they are not significant determinants of TCR recognition. Based on Parker matrix scores, the most favorable residue at each anchor site for each of the five alleles can be determined. Table 4 shows the resulting residue list by allele. To produce an analog peptide for a predicted epitope, the anchor residues of the predicted epitope can be changed to match the preferred residues for the same anchor sites of the allele being optimizing. In the event that the peptide already has the preferred residues in both primary anchor sites, an analog peptide would not be produced. In some embodiments, the binding affinities of the analog peptides and the original epitopes can be tested using the epitope prediction methods to confirm higher binding affinity for the analog.

TABLE 4
Most favored anchor resides by allele.
The preferred values are derived from BIMAS binding matrices
(http://bimas.dcrt.nih.gov/molbio/hla_bind/)
developed by the method of Parker et al. (1993).

| | Preferred Anchor Residues by Position | | | | | | | | |
|---|---|---|---|---|---|---|---|---|---|
| Allotype | 1 | 2 | 3 | 4 | 5 | 6 | 7 | 8 | 9 |

| | | | |
|---|---|---|---|
| A1 | | E | Y |
| A2 | L | | V |
| A3 | L | | K |
| A24 | Y | | L |
| B7 | P | | L |

H. Promiscuity

In theory, an peptide epitope for a given class and allele that is surrounded, in the sequence of the protein from which it was derived, by epitopes for different classes and alleles, is more likely to initiate an actual immune response than epitope that is not. In some embodiments of the present teachings, the methods of finding and catagorizing epitopes has been extended to seek out proteins that contain high densities of peptide epitope sequences. These methods can be constructed to find promiscuous epitopes or epitope-dense regions of a protein. In some embodiments, for a given k-mer protein, the number of epitopes it contains is counted. Proteins containing higher numbers of epitopes are selected for further investigation and analysis.

I. Definitions

Unless defined otherwise, technical and scientific terms used herein have the same meaning as commonly understood by one of ordinary skill in the art to which the present teachings belong. One skilled in the art will recognize many methods and materials similar or equivalent to those described herein, which can be used in the practice of the present teachings. Indeed, the teachings presented are in no way limited to the methods and materials described. For purposes of the present teachings, the following terms are defined below.

As used herein, “binding” is defined as an association between molecules. In some embodiments of the present teachings, the “binding affinity” of a peptide for a protein refers to the likelyhood that a peptide would associate with a protein. Multiple units, measures and labels may be used to characterize binding affinity. Binding affinity may be estimated or measured using qualitative or quantitative terms.

The terms “prediction” and “predicting” refer to the use of the present teachings to estimate properties of molecules under consideration. The use of the present teachings to estimate the properties of peptides can be done to peptides of known or unknown properties.

“MHC” refers to the Major Histocompatibility Complex, its proteins and genetic elements. “Human protein” and “viral protein” refer to any peptide encoded by a genetic element (e.g. DNA and/or RNA) originating from the genome of a human or a virus. These proteins may be fragments, parts or subunits of functional proteins.

As used herein, “Combining” refers to the adding together in some fashion of individual estimations. In some embodiments of the present teachings, combining will produce a single estimation from multiple estimations. In some embodiments of the present teachings, combining will create a reduction in the number of estimations in a set of estimations.

“Voting” refers to the addition of information from a method to a combined estimation. In some embodiments of the present teachings, voting results in a merger of estimations. In some embodiments of the present teachings, the results of voting fall on a continuum or a range of values that may be assigned to molecules. In some embodiments of the present teachings, voting creates a decision about the properties of a molecule, e.g., whether a peptide belongs to a category or not.

“Sequence data” refers to information about the amino acid residues that constitute a peptide or protein, or the genetic or nucleic acid information that is related to the protein or peptide.

As used herein, the storage of information in a centralized database is understood to be the containment of information within a single database or with a connected system incorporating a database or databases.

The manufacture of a peptide is defined as the creation of a peptide through a variety of means, these means including but not limited to: digestion, degradation or hydrolysis of a protein; assembly of amino acids in vitro; and the harvesting of peptides from organisms, cells or biological sources.

As used herein, the adjective “tumor” can describe proteins, peptides, genetic elements, and any molecular or biological substance isolated, derived, associated with or identified as originating in or on a malignant cell, malignant mass or any type of neoplasm. A “viral gene” is a genetic sequence with at least a partial origin in the genome, or expressed RNA from the genome, of a virus or viral element.

A “computer system” refers to an electronic computational system for performing a method of the present teachings. Some embodiments of the present teachings include a computer system for carrying out a method of the present teachings. In some embodiments, a computer system can be involved in, but is not limited to being involved in, the creation of peptide sequences for consideration by the methods of the present teachings; any methods of the present teachings; the storage and/or manipulation of the products of the methods of the present teachings; and the presentation of the products of the methods of the present teachings. The system may be contained within one location or may have parts in various locations. The parts of the system may be connected by a computer network.

### III. EXAMPLES

Material and Methods

For the QP method, a set of 101 HLA-A2 epitopes along with their IC-50 level binding information were extracted from public databases (Parker et al., 1 994a). A set of 694 ninemer epitopes where extracted from the MHCPEP database (http://wehih.wehi.edu.au/mhcpep/; Brusic et al., 1998). Of these epitopes, 359 were annotated with their binding strength categories (high, medium or low).

For the LP method, allele-specific ninemer data were extracted from the same MHCPEP database. For the HLA-A2 allele, 692 epitopes were used, 85 for A1, 118 for A3, 23 for A24, and 56 for B7.

For both the QP and LP methods, initial predictions were done for ninemers. Tenmer scores were then computed by ignoring the seventh position in the sequence.

For the profile-based methods, epitope and ligand sequences previously published for the HLA-A0201 allele were extracted from the SYFPEITHI database (http://svlpeithi.bmi-heidelberg.com; Rammensee et al., 1999). Similarly, data for the HLA-A1, A3, A11, A24 and B7 alleles were extracted from the MHCPEP database. After eliminating duplicates and sequences with more or less than nine residues from the 206 HLA-A0201 ligands and epitopes, the remaining 146 distinct ninemers were selected for profile construction. For HLA-A2 anchor scoring, the entire pool of peptides, regardless of length, was analyzed to determine the frequencies of amino-acid pairs at the P2 and C-terminal positions. The procedure for the other alleles was similar.

Results for Epitope Pipeline Prediction Method

Graphs of sensitivity curves were used to measure and compare the performance of the various epitope prediction methods tested. For example, consider a benchmark consisting of a protein, or a set of peptide sequences, for which all epitopes have been previously identified, and a prediction method that ranks all peptides by their scores. The sensitivity curve plots the sensitivity Sn(x)=TP/(TP+FN) achieved by the method when the top ranking x% of the peptides are selected and classified as epitopes, where TP, TN, FP, FN are the numbers of true positive, true negative, false positive and false negative examples in the reference set. In other words, a sensitivity curve plots the percentage ƒ of epitopes that are found in the top ranking x% of the peptides. The answer to the question “What percentage of the peptides needs to be sequenced and tested in order to obtain f% of the epitopes in the pool?” can then be easily read from the horizontal axis of the graph.

Sensitivity curves are commonly summarized by examining a small number of critical values. For example, the fraction of top-ranking peptides that must be synthesized to cover 25%, 50%, 75%, and 100% of all true epitopes can be examined.

In the example below, the algorithms are benchmarked with a reference set of known epitopes and MHC ligand sequences, collected from the literature and from publicly accessible databases. The predictions are compared to those produced with an improved version of the matrix-based approach presented in (Parker et al., 1993), available from the NIH BIMAS site (http://bimas.dcrt.nih.gov/molbio/hla_bind/).

### Example 1

### Cancer Benchmark

The focus of some embodiments is to apply methods of the present teachings to cancer immunotherapeutics. To this end, a benchmark set consisting specifically of epitopes isolated from human cancers was created. The intention was to test whether methods trained on a general epitopes set would be equally effective on epitopes specifically isolated from cancers. Cancer epitopes might reasonably be expected to exhibit some selective biases, as they are generally expressed in self-proteins and therefore would presumably have to escape immune tolerance in addition to the other selectivity constraints presented on epitopes from foreign antigens. The experiment first used a collection of epitopes reported by Renkvist et al. (Renkvist et al., 2001) that were isolated from human tumor cells. Those epitopes that do not occur in the wild-type versions of their respective proteins were screened out. This screening was conducted by using only those epitopes that could be found in proteins in the Celera predicted gene translations (a set of protein sequences predicted from the Celera human genome sequence) or those that could be found in the Genbank (Benson et al., 2000), SwissProt/TrEMBL (Bairoch and Apwieler, 1997), or PIR (Wu et al., 2002) databases. The search was further confined to epitopes of length 9 or 10 for the HLA-A1, HLA-A2, HLA-A3, HLA-A24, and HLA-B7 class I alleles . The result was a set of 70 epitopes from 30 proteins.

The benchmark test consisted of ranking peptides from the complete set of proteins from which the epitopes were derived. Sensitivity curves were then plotted as described above, measuring the effectiveness of the methods in ranking true epitopes high among the list of peptides ranked by predicted binding affinity. Table 5 shows the sensitivity curves for the cancer benchmark for one embodiment of a combined voting method of the present teachings (red) and for the reference method (blue) for the A2 alleles. Table 6 shows results for all five alleles examined.

[image]

TABLE 6
Critical values comparing a voting method of the present teachings
to the reference method matrices for the cancer benchmark. For each
allele, the percentage of top ranking peptides needed to contain
25%, 50%, 75%, and 100% of all true epitopes is given in the data set

| Allele | Method | 25% | 50% | 75% | 100% |
|---|---|---|---|---|---|
| A1 | Voting | 0.0032% | 0.0063% | 0.35% | 0.95% |
| | Parker | 0.019% | 0.022% | 0.35% | 3.2% |
| A2 | Voting | 0.46% | 1.5% | 3.2% | 40% |
| | Parker | 0.27% | 0.85% | 3.2% | 53% |
| A3 | Voting | 0.085% | 0.13% | 0.18% | 0.59% |
| | Parker | 0.0095% | 0.24% | 0.41% | 0.53% |
| A24 | Voting | 0.14% | 0.45% | 4.3% | 6.9% |
| | Parker | 0.20% | 0.45% | 0.80% | 12% |
| B7 | Voting | 3.5% | 3.5% | 3.5% | 3.5% |
| | Parker | 7.7% | 7.7% | 7.7% | 7.7% |

The sensitivity curves (FIG. 5) show the differences in the performance between a method of the present teachings and the Parker method, with the method of the present teachings performing better on the majority of the sections of the epitope range and performing consistently better when more complete sets are sought. While the Parker method may be better locating the strongest binders, our method is superior at identifying those binders that are placed towards the mid and lower ranges of the binding affinity scale. This property is particularly desirable for developing cancer vaccines, in which high-binding self-peptides may lead to host tolerance. Because high-binders would be less selective in inducing an immune response, peptides that bind in the mid and lower ranges of the binding affinity scale would be the most desirable for cancer vaccines. Furthermore, the ability to efficiently predict a range of peptides containing as complete a set of epitopes as possible given reasonable sequencing costs would strongly benefit high-throughput vaccine development.

### IV. Conclusions

The computational pipeline that is incorporated by some embodiments of the present teachings creates a unified method for integrating diverse lines of evidence into the prediction of T-cell epitopes. In some embodiments, the pipeline offers integrated methods of isolated prediction methods that are computationally tractable for large-scale epitope predictions. In some embodiments, the pipeline also integrates subtasks of the epitope prediction problem, including binding level prediction, analog design, and the detection of regions of high promiscuity. Hence some embodiments feature a pipeline that establishes a methodology for the complete selection of peptides for T-cell based vaccines targeted at specific proteins, taking the process from target protein amino acid sequences through the selection of observed peptides or their analogs for vaccine construction.

Additional embodiments of the present teachings take advantage of the modular nature of the pipeline. As additional singular methodologies are developed for predicting the binding of a peptide to a molecule, those methodologies can be added to the pipeline and may enhance its performance. For example, some embodiments of the present teachings incorporate structural-based methods that are less dependent on the existence of extensive data sets to build a model. Other embodiments incorporate computational learning modules in order to reduce the space of models being examined, which can simplify and accelerate the prediction process.

Embodiments of the present teachings envision a variety of uses for a high-throughput epitope prediction pipeline. In one embodiment, application of the pipeline is related to the goal of cancer immunotherapeutics, as part of a broader high-throughput approach to target identification followed by vaccine development. In some embodiments, rapid high-throughput screening of epitopes is used for accelerated development of vaccines to novel strains of known pathogens; this can be done in conjunction with high-throughput sequencing, to rapidly develop vaccines to previously unknown pathogens. In other embodiments, the results of high-throughput scans of whole genomes are used to reveal potential causes of autoimmune diseases. In addition to medically directed purposes, other embodiments direct the predictive power of the methods discussed herein towards basic research areas, such as comparative genomics.

What is claimed is:

1. A method for predicting the binding affinity of a peptide for a MHC protein, comprising providing a prediction of binding affinity by two or more methods, and then combining the predictions of those methods.

2. The method of claim 1, wherein said MHC protein is a MHC class I protein.

3. The method of claim 2, wherein said MHC class I protein is an allotype selected from the group consisting of A1, A2, A3, A24, and B7.

4. The method of claim 1 wherein said peptide is derived from a protein selected from the group consisting of a viral protein and a human protein.

5. The method of claim 1, wherein the sequence of said peptide is derived from a sequence selected from the group consisting of a sequence from a known protein or a genomic sequence.

6. The method of claim 1, wherein said combining is performed by a voting method.

7. The method of claim 1, wherein said predictions of affinity are presented in terms selected from the group consisting of relative binding efficiencies, IC-50 values, and categorical binding affinities.

8. The method of claim 1, wherein said methods are algorithms selected from the group consisting of quadratic programming, linear programming, and a profile-based method.

9. The method of claim 8, wherein said profile-based method uses a clustering heuristic selected from the group consisting of iterative multiple alignment, letter frequencies, and position dependencies reflected by (2 tests.

10. The method of claim 8, wherein said profile-based method employs a priniciple selected from the group consisting of dimensionality reduction, multiple intra-allelic motifs, and anchor selection.

11. A computer system, comprising programming to perform the method of claim 1.

12. A computer storage media, comprising programming to perform the method of claim 1.

13. A method for assigning a categorical binding affinity for a protein to a candidate peptide based on information for a set of known peptides for a protein, comprising:
obtaining sequence and binding affinity information for a set of known peptides;
obtaining sequence data for a candidate peptide;
generating an evaluation of the affinity of said candidate peptide for said protein using said sequence and binding affinity information for a set of known peptides and said sequence data for candidate peptides;
ranking said candidate peptide and said known peptides on the basis of relative binding affinities for said protein; and
assigning peptides to categories of binding affinity based on rank.

14. The method of claim 13,wherein ranking comprises a list of said known peptides organized by their affinities for said protein.

15. The method of claim 13, wherein said ranking includes said candidate peptide.

16. The method of claim 15, wherein said categorical binding affinity of said candidate peptide is expressed as assignment of the peptide to one of two or more classes based on said ranking of said candidate peptide in said list of individual peptides from said set of known peptides.

17. The method of claim 16, wherein said classes of categorical binding affinity have labels selected from the group consisting of high, medium, low and non-binder.

18. A method for evaluating the affinity of a candidate peptide for a protein, consisting of:
obtaining sequence and binding affinity information for a set of known peptides;
obtaining sequence data for a candidate peptide;
generating a first evaluation of affinity of said candidate peptide for said protein by comparing the sequence of said candidate peptide with data developed from said information for a set of known peptides with a first prediction method;
generating one or more additional evaluations of affinity of said candidate peptide for said protein using said information for a set of known peptides and one or more additional prediction methods different from said first method used to generate said first evaluation of affinity; and
combining said first evaluation and said one or more additional evaluations into a combined evaluation.

19. The method of claim 18, wherein said binding affinity information for a set of known peptides comprises data selected from the group consisting of quantified affinity data for individual peptides in the set, data regarding whether the individual peptides bind said protein or not, data comprising the assignments of known peptides into two or more binding categories, and data that combines two or more of these data types.

20. The method of claim 19, wherein said data comprising the assignment of known peptides into two or more binding catagories comprises assignment of peptides to high, medium, low, and non-binder categories.

21. The method of claim 18, wherein said prediction method and said one or more additional prediction methods are selected from the group consisting of quadratic programming, linear programming, and a profile-based method.

22. The method of claim 21, wherein said profile-based method incorporates a method selected from the group consisting of iterative multiple alignment, letter frequencies, and position dependencies reflected by χ² tests.

23. The method of claim 21, wherein one prediction method selected is quadratic programming.

24. The method of claim 23, wherein a data reduction method, selected from the group consisting of BIMAS-like method and AA-properties method, is used with quadratic programming.

25. The method of claim 21, wherein said profile-based method implements a principle selected from the group consisting of dimensionality reduction, multiple intra-allelic motifs, and anchor selection.

26. The method of claim 18, wherein said sequence data for a candidate peptide is selected from the group consisting of data derived from the sequence data of a known protein and data derived from the sequence data of an organism.

27. The method of claim 18, wherein said sequence data for a candidate peptide is selected from the group consisting of viral protein sequence data and human protein sequence data.

28. The method of claim 18, wherein said sequence data for a candidate peptide is stored in a centralized database.

29. The method of claim 18, wherein said sequence and binding affinity information for a set of known peptides is stored in a centralized database.

30. The method of claim 18, wherein said first evaluation is stored in a centralized database.

31. The method of claim 18, wherein said one or more additional evaluations is stored in a centralized database

32. The method of claim 18, wherein said combined evaluation is stored in a centralized database.

33. The method of claim 18, wherein said protein is a MHC protein.

34. The method of claim 33, wherein said MHC protein is a MHC class I protein.

35. The method of claim 34, wherein said MHC class I protein is an allotype selected from the group consisting of A1, A2, A3, A24, and B7.

36. The method of claim 18, wherein said combined evaluation comprises assignment of a rank to said candidate peptide based on said candidate peptide's affinity to said protein.

37. The method of claim 36, wherein said rank based on said candidate peptide's affinity to a protein is a ranking relative to other peptides.

38. The method of claim 36, wherein said combined evaluation comprises assignment of said candidate peptide to a class, according to said rank based on its affinity to said protein.

39. The method of claim 18, further comprising the evaluation of additional candidate peptides.

40. The method of claim 39, further comprising the selection of a subset of candidate peptides based on the combined evaluations of those peptides.

41. A computer system, comprising programming to perform the method of claim 18.

42. A computer storage media, comprising programming to perform the method of claim 18.

43. A method for making a vaccine, comprising manufacturing a peptide selected using the method of claim 18 and preparing a medicament containing the selected peptide.

44. An algorithmic quadratic programming (QP) method, comprising methods for predicting the relative affinities of peptides proteins.

45. The method of claim 44, further comprising the equation and constraints:
$${min}_{w , c} {\sum }_{i} {( {x}_{i}^{T} w - c - {b}_{i} )}^{2}$$
$$s \cdot t \cdot {x}_{{H}_{i}}^{T} w \geq {x}_{{M}_{j}}^{T} w ∀ i , j , \\ {x}_{Hi}^{T} w \geq {x}_{Lj}^{T} w ∀ i , j , \\ {x}_{{M}_{i}}^{T} w \geq {x}_{Lj}^{T} w ∀ i , j , \\ {x}_{{e}_{i}}^{T} w \geq {IC}_{min}^{50} , {x}_{{ne}_{i}}^{T} w \leq {IC}_{min}^{50}$$

46. The method of claim 44, further comprising the equation and constraints:
$$\begin{matrix}\begin{matrix}{min}_{w , c} {\sum }_{i} {( {x}_{i}^{T} w - c - {b}_{i} )}^{2} + {\sum }_{i , j} {e}_{{H}_{i} {H}_{j}}^{2} + {\sum }_{i , j} {e}_{{H}_{i} {L}_{j}}^{2} + \\ {\sum }_{i , j} {e}_{{M}_{i} {L}_{j}}^{2} + {\sum }_{i} {e}_{{e}_{i}}^{2} + {\sum }_{i} {e}_{{ne}_{i}}^{2}\end{matrix} \\ s \cdot t \cdot ( {x}_{{H}_{i}}^{T} - {x}_{{M}_{j}}^{T} ) w + {e}_{{H}_{i} {M}_{j}} \geq 0 ∀ i , j \\ ( {x}_{{H}_{i}}^{T} - {x}_{{L}_{j}}^{T} ) w + {e}_{{H}_{i} {L}_{j}} \geq 0 ∀ i , j \\ ( {x}_{{M}_{i}}^{T} - {x}_{{L}_{j}}^{T} ) w + {e}_{{M}_{i} {L}_{j}} \geq 0 ∀ i , j \\ {x}_{{e}_{i}}^{T} - {IC}_{min}^{50} + {e}_{{e}_{i}} \geq 0 ∀ i \\ {IC}_{min}^{50} - {x}_{{ne}_{i}}^{T} w + {e}_{{ne}_{i}} \geq 0 ∀ i\end{matrix}$$

47. A linear programming method of predicting peptide affinity for a protein.

48. The method of claim 47, further comprising the equation and constraint:
$$\begin{matrix}min {\sum }_{aa ' s a} {\sum }_{pos \cdot p} {b}_{ap} \\ s \cdot t \cdot {\sum }_{pos \cdot p} {b}_{{e}_{i}^{p} p} \geq 1 ∀ i \\ {b}_{ap} \geq 0 ∀ a , p\end{matrix}$$

49. A profile-based method of predicting peptide affinity for a protein, comprising:
obtaining information for a set of known peptides;
creating one or more motifs for peptides with affinity to said protein by analyzing said information for a set of known peptides; and
evaluating the affinity of a candidate peptide based on said one or more motifs.

50. The method of claim 49, wherein said information comprises sequence information for said set of known peptides.

51. The method of claim 49, further comprising a clustering heuristic selected from the group consisting of iterative multiple alignment, letter frequencies, and position dependencies reflected by χ² tests.

52. An iterative multiple alignment (aln) method of predicting the relative binding affinity of a peptide for a MHC protein, comprising:
obtaining sequence and affinity information for a set of known epitopes for said MHC protein;
deriving a motif from said information for a set of known epitopes via an iterative multiple alignment heuristic;
generating a score for said peptide based on its similarity to said motif; and
predicting the relative binding affinity of said peptide for said MHC protein based on said score.

53. The method of claim 52, further comprising:
deriving multiple motifs from a set of known epitopes via an iterative multiple alignment heuristic;
generating scores for said peptide, each of said scores based on its similarity to one of said motifs; and
predicting the relative binding affinity of said peptide for said MHC protein based on the score showing the highest binding affinity.

54. A letter frequency (LetFq) method of predicting the relative binding affinity of a peptide for a MHC protein, comprising:
obtaining sequence and affinity information for a set of known epitopes for said MHC protein;
deriving a motif from said information for a set of known epitopes via a letter frequency heuristic;
generating a score for said peptide based on its similarity to said motif; and
predicting the relative binding affinity of said peptide for said MHC protein based on said score.

55. The method of 54, further comprising:
deriving multiple motifs from a set of known epitopes via a letter frequency heuristic;
generating scores for said peptide, each of said scores based on its similarity to one of said motifs; and
predicting the relative binding affinity of said peptide for said MHC protein based on the selection of the score showing the highest binding affinity.

56. A method based on χ² statistical significance (Ki2) tests predicting the relative binding affinity of a peptide for a MHC protein, comprising:
obtaining sequence and affinity information for a set of known epitopes for said MHC protein;
deriving a motif from said information for a set of known epitopes according to dependencies between peptide positions, revealed by (i2 significance tests;
generating a score for said peptide based on its similarity to said motif; and
predicting the relative binding affinity of said peptide for said MHC protein

57. The method of claim 56, further comprising:
deriving multiple motifs from a set of known epitopes via analysis of position dependencies with χ² significance tests
generating scores for said peptide, each of said scores based on its similarity to one of said motifs; and
predicting the relative binding affinity of said peptide for said MHC protein based on the selection of the score showing the highest binding affinity.

58. A method of selecting peptides with a desired level of affinity for a protein, comprising:
obtaining sequence data for a set of candidate peptides and data for peptides of known affinity for said protein known peptides;
evaluating the affinity of said candidate peptides for said protein using two or more methods, at least one of which uses the data for the peptides of known affinity; and
selecting peptides from said set of candidate peptides that are predicted by said two or more methods to have said desired level of affinity for said protein.

59. The method of claim 58, wherein said data for peptides of known affinity comprises: a) sequence data and b) data selected from the group consisting of quantitative binding affinity data and qualitative binding data.

60. The method of claim 58, further comprising using said peptides with a desired level of affinity for a protein for the treatment or prevention of disease.

61. A method for providing treatment for a patient with, or at risk for, cancer or a viral infection, comprising:
obtaining sequence data for candidate peptides;
obtaining data for a set of known peptides;
evaluating the affinity of said candidate peptides for a protein with two or more prediction methods;
selecting candidate peptides with a desired level of affinity for the protein for inclusion in a treatment; and
treating said patient by stimulating an immune response in said patient with said treatment.

62. The method of claim 61, wherein said sequence data for candidate peptides is selected from the group consisting of sequence data derived from tumor cell proteins, sequence data derived from tumor cell genes, sequence data derived from viral proteins, and sequence data derived from viral genes.

63. The method of claim 61, wherein said data for a set of known peptides comprises: a) sequence data and b) data regarding the affinity of said known peptides for said protein.

64. The method of claim 61, further comprising a step of determining the allotype of said protein, by obtaining genetic information about said patient.

65. The method of claim 64, wherein said protein is a MHC protein.

66. A method for screening a set of candidate peptides, comprising:
obtaining sequence data for candidate peptides;
obtaining data for a set of known peptides;
evaluating the affinity of said candidate peptides for a protein with two or more prediction methods;
assigning said candidate peptides to two or more groups based on the evaluations of affinity; and
screening said set of candidate peptides on the basis of group assignment.

67. The method of claim 66, wherein said sequence data for candidate peptides is selected from the group consisting of sequence data of a known protein, sequences of a set of known proteins, and genomic sequences.

68. The method of claim 67, wherein said sequence data for candidate peptides is generated by dividing the sequence data of a known protein into ninemer or tenmer fragments.

69. The method of claim 66, wherein said data for a set of known peptides comprises: a) sequence data and b) data regarding the affinity of said known peptides for said protein.

70. A method for identifying epitopes, comprising:
obtaining sequence data for candidate peptides;
obtaining data for a set of known peptide epitopes that bind a MHC protein;
evaluating the affinities of said candidate peptides for said MHC protein using two or more prediction methods and said data for a set of known peptide epitopes;
assigning said candidate peptides to two or more groups based on the evaluations of affinity; and
identifying a set of said candidate peptides by group assignment.

71. The method of claim 70, wherein said epitopes are T-cell epitopes.

72. The method of claim 70, wherein said MHC protein is MHC class I protein.

73. The method of claim 72, wherein said MHC class I protein is an allotype selected from the group consisting of A1, A2, A3, A24, and B7.

74. The method of claim 70, wherein said two or more prediction methods are selected from the group consisting of quadratic programming, linear programming, and a profile-based method.

75. The method of claim 74, wherein said profile-based method incorporates a method selected from the group consisting of iterative multiple alignment, letter frequencies, and position dependencies reflected by (2 tests.

76. The method of claim 74, wherein one prediction method selected is quadratic programming.

77. The method of claim 76, wherein a data reduction method, selected from the group consisting of BIMAS-like method and AA-properties method, is used with quadratic programming.

78. The method of claim 70, wherein said evaluations comprise assignment of said candidate peptides and said known peptide epitopes to a rank based on their relative affinity to said MHC protein.

79. A computer system for predicting the affinity of candidate peptides for a protein, comprising:
a database containing data for sets of known peptides;
a database containing sequence information for said candidate peptides;
a processor capable of evaluating peptides by multiple methods; and
a database for storing evaluations.

80. The system of claim 79, further comprising programming for the execution of said multiple methods of analyzing said candidate peptides.

81. The system of claim 80, wherein said programming further comprises methods of combining peptide evaluations into a composite evaluation.

82. The system of claim 79, wherein said evaluations are predictions of affinity of said candidate peptides for a protein.

83. The system of claim 79, wherein said data for sets of known peptides comprises: a) sequence data and b) data regarding the affinity of said known peptides for a protein.

84. The system of claim 79, wherein said sets of known peptides comprise peptides that are epitopes with affinity for or recognized by immune system proteins.
